(19) United States
(12) Patent Application Publication
(10) Pub. No.: US 2020/0168299 A1
(43) Pub. Date: May 28, 2020

(54) SYSTEMS AND METHODS FOR TARGETED GENOME EDITING

(71) Applicant: PIONEER HI-BRED INTERNATIONAL, INC., JOHNSTON, IA (US)

(72) Inventors: ANDREW BAUMGARTEN, JOHNSTON, IA (US); JUSTIN P GERKE, URBANDALE, IA (US); HUI GUO, JOHNSTON, IA (US); MATTHEW G KING, JOHNSTON, IA (US); HAINING LIN, CLIVE, IA (US); ROBERT B MEELEY, DES MOINES, IA (US); BROOKE PETERSON-BURCH, ANKENY, IA (US); YUN ZHANG, JOHNSTON, IA (US)

(73) Assignee: PIONEER HI-BRED INTERNATIONAL, INC., JOHNSTON, IA (US)

(21) Appl. No.: 16/632,899

(22) PCT Filed: Jul. 27, 2018
(86) PCT No.: PCT/US18/44112

§ 371 Date: Jan. 22, 2020

Related U.S. Application Data
(60) Provisional application No. 62/538,213, filed on Jul. 28, 2017.
(60) Provisional application No. 62/573,402, filed on Oct. 17, 2017.

(51) Int. Cl. G16B 30/20; G16B 30/10; G16B 20/20; G06F 30/20

(52) U.S. Cl. CPC G16B 30/20; G06F 30/20; G16B 20/20; G16B 30/10

(57) ABSTRACT

Systems and methods are described for designing nucleotide guides for site-specific genome editing that also minimize off-target genome edits. Systems and methods are described for using these nucleotide guides to edit specific genomic regions and minimize edits to genomic regions not intended for editing.

### CROSS-REFERENCE SECTION

This patent application claims priority to U.S. provisional patent application No. 62/573,402, filed on Oct. 17, 2017, and to U.S. provisional patent application No. 62/538,213, filed on Jul. 28, 2017, the entire contents of which are hereby incorporated herein by reference.

### REFERENCE TO SEQUENCE LISTING SUBMITTED ELECTRONICALLY

The official copy of the sequence listing is submitted electronically via EFS-Web as an ASCII formatted sequence listing with a file named “7452WOPCT_Sequence_Listing_ST25” created on Jul. 25, 2018, and having a size of 33 kilobytes and is filed concurrently with the specification. The sequence listing contained in this ASCII formatted document is part of the specification and is herein incorporated by reference in its entirety.

### BACKGROUND

Recent developments in genome editing techniques have enabled sequence modifications of specific sequence locations. For example, sequence editing using CRISPR-Cas systems uses RNA complementary to a targeted DNA sequence to guide Cas proteins to specific sequence sites for modification, where a site is a sequence or a region within a sequence which is a natural or modified or artificial nucleic acid molecule or its representation. Editing experiments can include site-specific nucleases, such as CRISPR-Cas9, TALENs, meganucleases, targeted or tethered nucleases, programmable nucleases, Ribonucleoproteins (RNP) and may involve direct transformation, biolistic delivery, co-cultivation, or any number of delivery methods in order to achieve the specific, directed nucleic acid modification or edit. Such genome edits can be used to deliver genome modifications that confer desirable phenotypes, such as the improvement of agronomic traits in crop species.

### SUMMARY

Specific varieties, inbreds, or germplasm can be edited directly using any combination of methods to deliver genome editing components to plants or plant cells and then enriched or selected for the desired modification(s). Typically the varieties, inbreds, or germplasm will contain DNA sequence variation throughout the genome. Each distinct pattern of DNA sequence variation at two or more DNA base pairs is referred to as a haplotype. Knowledge of the haplotypes surrounding the location to be modified is required for each variety, inbred, or germplasm being subjected to editing in order to correctly target guide RNA or other reagents to the editing site and also to produce the desired sequence modification(s). So-called Trait Introgression (TI) or selective breeding introgression methods can be used to move an edited trait from one donor variety, inbred, or germplasm as a destination into a new variety, inbred, or germplasm. This is typically done via sexual propagation, but is not exclusive to sexually propagated crops. In TI, the typical process of enriching a targeted or selected introgression is via backcrossing strategies that monitor and select for the trait or molecular characteristic of interest, while simultaneously or successively enriching for a reasonable maximum percentage of the recurrent parent (destination) genome. Knowledge of the haplotypes harbored by the plant breeding population surrounding the target locus enables the selection of donor and recipient parents that minimize the genetic differences at the target locus, thus facilitating more rapid and accurate trait introgression. Novel traits, alleles, or molecular characteristics created by genome editing could be used in so-called Forward Breeding applications, where a genome edited line is a parent in breeding crosses with a set of additional varieties, inbreds, or germplasms to propagate and increase the frequency of the desired modification among the breeding population. To reduce the loss of genetic variation near the target locus, it may be desirable to make the edit in a set of genetic entities that represent all existing haplotypes in the larger population at the target locus. Such an approach would require knowledge of all sequence variation within the desired region.

Across all possible methods for the deployment of genome editing into novel varieties, inbreds, hybrids, germplasm, or products, it is desirable to have flexible methodologies that allow target- or allele-, or haplotype-specific or other such context-specific designs of the needed targeting components, or that allow conserved, preserved, identical, or generic methods of designs for the needed targeting components that may serve more broadly across a range of varieties, inbreds, hybrids, or germplasm, or even across sub- or species boundaries or sequence sets.

Another problem common to sequence editing techniques is that sometimes, in cases where the guide RNA or targeting nucleic acid component or other targeting component is not specific enough to the targeted site, it may guide editing to unintended, non-targeted (off-target) sequence regions, sometimes leading to undesired effects.

There is thus a need in the art for flexible nucleic-acid sequence editing systems and methods that accommodate sequence edits targeted to specific sites or groups of sites which take into account allelic similarities or differences and strategies, systems and methods that may also minimize unintentional off-target edits.

Disclosed herein are methods of designing a guide polynucleotide that minimizes the potential of generating off-target site gene edits. The methods may include (a) comparing a target site sequence for an endonuclease against unassembled raw nucleotide sequence reads from individuals in a population, (b) assembling the raw nucleotide sequence reads that align with part or all of the target site sequence into individual contigs, (c) selecting the target site sequence comprising a single copy of the target sequence in the contigs from step b, optionally, (d) designing a guideRNA for that target site sequence, and (e) generating an intended gene edit at the target site in a nucleic acid using the designed guide polynucleotide in an endonuclease complex.

Also disclosed herein are methods of creating a consensus sequence for a haplotype found in a population. The methods may include (a) sequencing a region of interest of two or more individuals of differing genotypes in a population to produce nucleotide sequence reads, (b) aligning the nucleotide sequence reads to one or more subject sequences to identify nucleotide variations, (c) using the nucleotide variations in the region of interest to define one or more haplotypes, (d) assigning at least one individual from the population to the one or more haplotypes in step (c), and (e) creating a haplotype consensus sequence assembled from the nucleotide sequence reads of the regions from the one or more individuals assigned in step (d).

Disclosed herein are methods of creating a consensus sequence for a subject haplotype found in a population. The methods may include (a) sequencing a region of interest of two or more individuals of differing genotypes in a population to produce nucleotide sequence reads, (b) aligning the nucleotide sequence reads to one or more subject sequences to identify nucleotide variations, (c) using the nucleotide variations in the region of interest to define one or more haplotypes, (d) assigning at least one individual from the population to the haplotypes in step (c), (e) creating a profile for nucleotide variant frequencies for each common haplotype based on the nucleotide variations in the region of interest to generate common haplotype profiles, (f) identifying whether there are breakpoints in the subject haplotype that correspond to the common haplotype profiles or combinations thereof, (g) assigning those regions of the subject haplotype defined by the breakpoints to the corresponding two or more common haplotypes, and (h) creating a consensus sequence for the haplotype assembled from the nucleotide sequence reads of the regions of the common haplotypes that the subject haplotype was assigned to from step (g).

Also disclosed herein are methods of characterizing two or more haplotypes found in a population. The methods may include (a) sequencing a defined region of interest in two or more individuals of differing genotypes in a population to produce nucleotide sequence reads, (b) using nucleotide variations in the defined region to define two or more haplotypes, (c) assembling the nucleotide sequence reads across the different genotypes into consensus sequences for the two or more haplotypes, (d) comparing the haplotype consensus sequences to identify one or more additional nucleotide variations, and (e) characterizing each haplotype based on the identified nucleotide variations in the region of interest. The methods may further include (f) assigning at least one individual from the population to one or more haplotypes based on the nucleotide variations, and (g) creating a haplotype consensus sequence assembled from the nucleotide sequence reads of the regions of the one or more individuals assigned, for example, in step (f).

### DESCRIPTIONS OF THE FIGURES

FIG. 1 provides an overview of the sequence context modelling algorithm with an example of 12 inbred lines. Various weighted and/or dashed lines mark the true haplotype relationships of the 12 inbred lines. The method leads to the creation of haplotype sequences referred to here as allele models.

FIG. 2 is a schematic diagram of the edit site selection process aspect of the invention for native abundance sequence sets.

FIG. 3 is a schematic diagram of the reference genome based site specificity screening process.

FIG. 4 is a schematic diagram of the reference free site specificity screening process.

FIG. 5 shows 10 identical-in-state groups parsed into major allele model groups within the SSS and NSS heterotic pools FIG. 6 provides an overview of how the methods of this invention are used for product development.

### DETAILED DESCRIPTION

The invention includes systems and methods for determination of the spectrum of nucleic acid sequences available to be acted upon by a sequence editing compound within a sequence collection. The invention additionally includes systems and methods for designing and/or selecting nucleic acid sequences that can specifically target regions of a sequence or collection of sequences to be edited, including, but not limited to genomes, while avoiding modifications to off-target sites not intended for editing. The invention further includes systems and methods for using the aforementioned nucleic acid sequences to guide genome editing systems to specifically target regions of one or more nucleic acids to be edited while minimizing avoiding off-target sites not intended for editing.

The following describes methods for merging sequence data from different inbreds, varieties, or germplasm based on shared genetic information, identification and selection of edit sites, and the design of sequences specifically targeting sequence regions to be edited while minimizing or avoiding modification of off-target sites not intended for editing. While this description is made in terms of inbred maize lines, it should be understood that the same method may be used for designing site-specific targeting nucleic acids to target any other type of plant, animal, microbe, sequence, collection of sequences, or any other natural or artificial nucleic-acid based entity. Additionally, while some aspects of this description focus upon the use of the Cas9-based editing system as a specific but non-limiting example, it should be understood that these methods can also be used broadly with minor, obvious modifications for other targeted sequence editing compounds including but not limited to TALENs, meganucleases, targeted or tethered nucleases, programmable nucleases, Ribonucleoproteins (RNP), homing endonucleases or restriction enzymes, etc.

The term “consensus sequence” refers to any nucleotide sequence to which two or more individuals in a population have corresponding nucleotide sequences with a predetermined degree of homology in their genomes.

The term “reference sequence” refers to any nucleotide sequence assembled as a representative sequence of at least a portion of the genome of a population.

The term “subject sequence” refers to any nucleotide sequence in a database of nucleotide sequences.

The term “haplotype” refers to the genotype of any portion of the genome of an individual or the genotype of any portion of the genomes of a group of individuals sharing essentially the same genotype in that portion of their genomes.

The term “subject haplotype” refers to any haplotypes in a database of haplotypes.

The term “common haplotype” refers to a haplotype found in more than a predetermined percentage of individuals in a population.

The term “major haplotype” refers to the haplotype found in more individuals in a population than any other haplotype.

The term “rare haplotype” refers to a haplotype found in fewer than a predetermined percentage of individuals in a population.

The term “breakpoint” refers to a point in a nucleotide sequence in which the sequence changes from being homologous to a first haplotype to being homologous to a second haplotype.

The term “profile” refers to a description of the genotypes of individuals of the same haplotype, optionally including information such as genotype allele frequencies.

### Example of a General Sequence Editing Workflow as Applied to a Set of Maize Genome Sequences

### Sequencing Strategy

Whole genome sequencing is performed for a set of inbred lines representing the germplasm or genetic material of interest. Each inbred may be represented by a varying amount or ‘depth’ of sequence reads.

### Read Alignment and Variant Calling

Sequencing reads generated from the whole genome sequencing at various sequencing depths (for example, 30×, 20×, 3×) are aligned to reference sequences using Bowtie2 (Langmead et al. 2012). Many other alignment programs are available as well, and will be available to one skilled in the art. For example, these might include bwa (Li and Durbin 2009), bwa-mem (Li 2013), NovoAlign (novocraft.com), GEM (Marco-Sola et al. 2012), SOAP2 (Li et al. 2009), CUSHAW2 (Liu and Schmidt 2012), SeqAlto (Mu et al. 2012), Meta-aligner (Nashta-ali et al. 2017), et al. After reads are aligned to the reference sequence, single nucleotide polymorphisms (SNP) are called using Samtools (Li et al. 2009) and filtered based on minimum read coverage and minimum rate of homogeneity of alleles from reads within an individual. Other popular SNP calling programs are available: freebayes (Garrison and Marth 2012), UnifiedGenotyper and HaplotypeCaller in the GATK package (DePristo et al. 2011; Van der Auwera et al. 2013), Platypus (Rimmer et al. 2014), SOAPsnp (Li et al. 2009) as well as many others. Any suitable SNP calling method may be used.

In some alternatives, sequences may be organized in a manner that brings all points of shared similarity among sequences in the set together and marks locations of divergence, for example in sequence graph based models. In some versions of these structures, abundance may be tracked and/or the reliability of sequences may be improved as part of the process of sequence incorporation.

### Haplotype Group Assignment

A haplotype refers to a combination of alleles at more than one DNA sequence variant in a genomic region of interest. Genetic material can be assigned to haplotype groups for a sequence region. Haplotype groups can be defined as the set of genetic entities that carry the same alleles at the genetic variants present in the population at the region of interest. A preferred interpretation of a haplotype group is that members of the haplotype group share identical DNA sequence for the region. In some methods a haplotype group can be interpreted as a group of inbreds that share genetically related but non-identical DNA sequence for the genome region. The genetic entities in the haplotype groups can be inbred lines assigned to a single haplotype group. In some methods the genetic material can be heterozygous, such that some genetic entities can be assigned to two different haplotypes. In this case the individual haplotype groups can be determined or estimated from the heterozygous genotypes using pedigree information or the haplotypes of homozygous individuals in the population. The set of sequences used to define haplotypes and assign individuals to haplotype groups in the following set of example methods derive from maize genome sequences but it should be understood that they could in fact be any collection of sequences from any source, natural or otherwise, and the methods applied similarly, independent of the source sequence set type. Haplotype groups represent the spectrum of variants to consider for both intentional sequence modification targets as well as the possible range of off-target sites within the sequence set. Multiple published, peer-reviewed methods exist for creating haplotypes and will be available to one skilled in the art. Examples include BEAGLE (Browning and Browning 2007) and SHAPEIT (Deleaneu et al. 2013), et al. A haplotype group can be defined with respect to a specific sequence interval. In other methods a haplotype group can be extended along the genome for as long as the criteria of genetic identity or similarity are met. The measure for genetic identity or similarity can be based on SNPs, insertions and deletions, copy number variation, epigenetic marks, or a combination of these features or other sequence polymorphisms suitable for differentiating sequences in the set. In some methods a measure of genetic similarity or genetic identity may be based on sequence feature differences among the genetic entities. In some methods this score may be based on a count or frequency measure of the feature differences. Some methods may score heterozygous genotypes or missing data differently than a homozygous DNA sequence difference. Some methods may set thresholds for the allowable number or frequency of missing data and heterozygous genotypes. Some methods may weigh the score of a match or mismatch differently for different the allele frequencies of each allele in the full population of genetic entities. Some methods may estimate haplotype groups from the DNA sequence similarity using a probabilistic model. In some methods, the probabilistic model may include a model of the shared population history of the genetic entities, which may include pedigree information describing the familial relationships of the genetic entities. Such a model can also include information regarding expected haplotype frequencies, linkage disequilibrium, and patterns and rates of genetic recombination among haplotypes. In some methods a threshold may be set for assigning genetic entities to the same haplotype group. Thresholds can be based on the measure of genetic similarity or difference. The threshold can be based on an estimate of the probability that genetic entities share the same haplotype based on a probabilistic model.

In some methods, missing data may be imputed prior to haplotype assignment. Imputation is widely practiced by those skilled in the art. Some methods conduct imputation jointly with haplotype assignment. Other methods conduct imputation prior to haplotype assignment. Some methods conduct imputation for a genetic variant using only other variants within a specified genetic or physical distance in the genome. Other methods conduct imputation using all genetic loci on a single chromosome or across the entire genome. Some methods use a nearest neighbor approach, where imputation is informed by a different genetic entity with the lowest genetic distance from the genetic entity in question, given a measure of genetic distance. Some methods conduct imputation using information from all genetic entities within a specified genetic distance. In some methods the allele frequencies within the full population of genetic or nucleic acid entities may be used as information for imputation. In some methods, a probabilistic model may be used to conduct imputation. In some methods, the probabilistic model may include a model of the shared population history of the genetic entities, which may include pedigree information describing the familial relationships of the genetic entities. Such a model can also include information regarding expected allele frequencies, haplotype frequencies, linkage disequilibrium, and patterns and rates of genetic recombination among haplotypes.

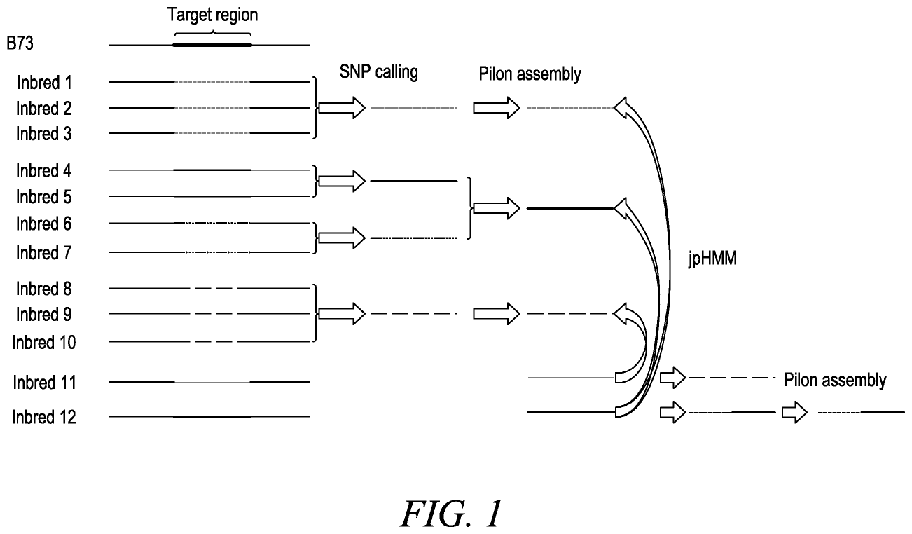

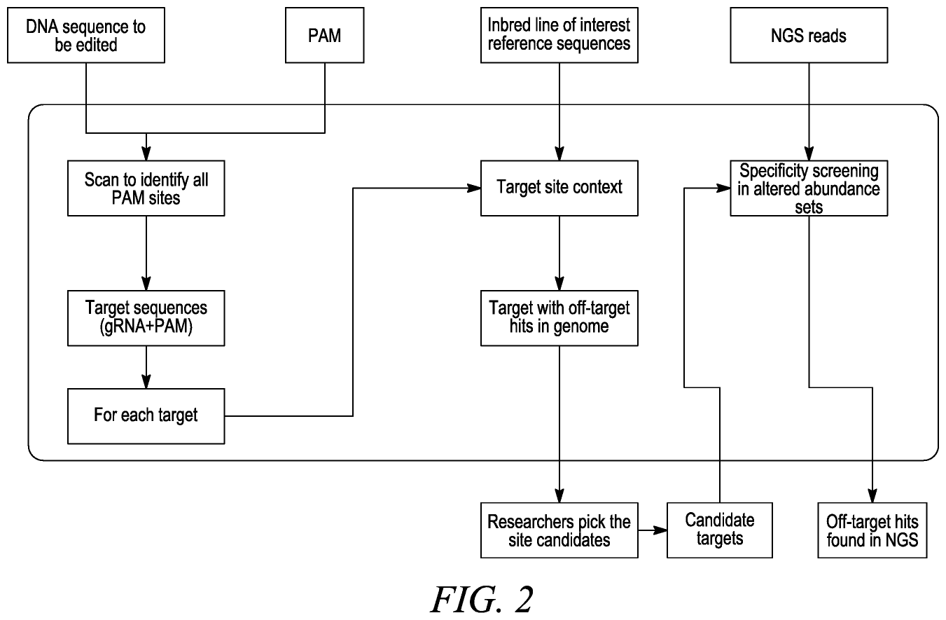

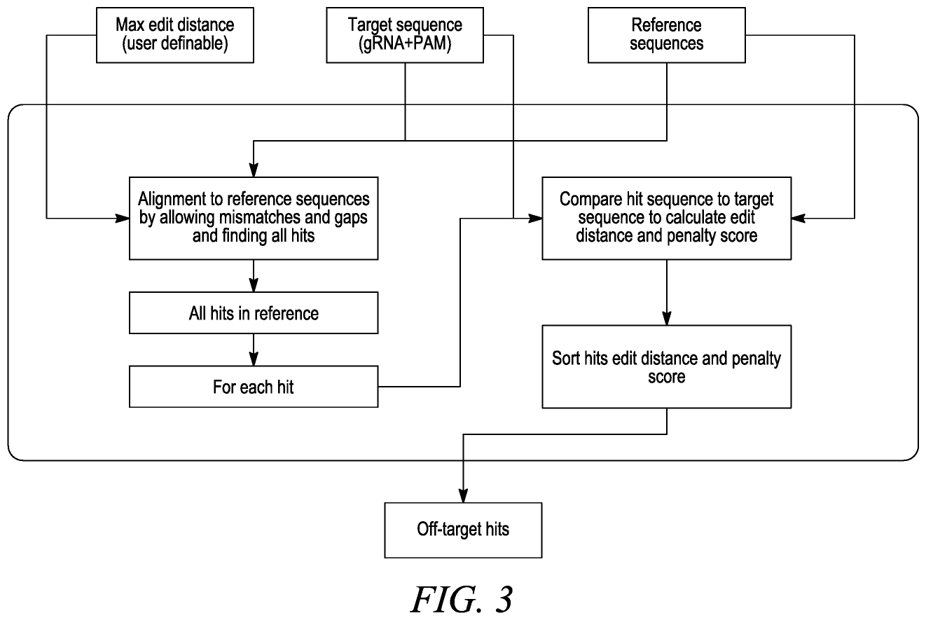

A haplotype group can be thought of as a cluster of genetic entities that share identical or similar DNA sequence within a specific genome region. The accuracy of haplotype clustering is largely affected by the prevalence and quality of SNPs identified in the target region or regions. Where the acronym “SNP” may be used for brevity, it should be understood that many other types of polymorphisms, as mentioned above, could be used instead. SNPs called from samples of low sequencing depth could result in low SNP density and a high level of missing data. In the method described herein, a two-round haplotype clustering method was used to mitigate this issue (FIG. 1). High quality SNPs from the target region plus 5′ and 3′ flanking regions (default 3kb) were used for the first round hierarchical clustering of inbred line sequences with a stringent identity threshold requirement (default 100%). If the number of SNPs was less than the desired threshold (default 20), the window was extended to flanking regions by incremental steps (default 1kb) until the threshold was met. Samples with the same haplotype in the target region were clustered into a haplotype group. A haplotype group with less than a given number of sources or inbred lines (default 3) was defined as a rare haplotype group. A haplotype group with membership equal to or greater than a certain number of sources or inbred lines (default 3) was defined as a major haplotype group and used in the next step for SNP calling. In this example, sequencing read alignments from sources in the same major haplotype group were merged into one BAM file for the target region. Pilon (Walker et al. 2014) and vcftools (Danecek et al. 2011) were used to call a set of new SNPs for each of the haplotype groups for the target region using the merged BAM files. In principle, other SNP calling methods (See the section of variant calling) can be used with the sequence information provided in any of a wide array of formats or approaches for this step as well. The new SNP (polymorphism) set, which may contain more or different SNPs than those used in the first round of haplotype clustering, was then used for the second-round clustering of the sources or inbred lines from the major haplotype groups identified above using the same clustering algorithms as the aforementioned haplotype assignment methods. Since this second set of SNPs may contain more information than the initial set, it can produce more accurate haplotype clusters while using a smaller window of the genome.

### Local Assembly for Major Haplotype Groups

For a given haplotype group defined for a given region of interest, there can be multiple genotypes sequenced at different sequencing levels, e.g. 3×, 30×, 100×, or higher, or less. Since all the genotypes in the haplotype group share the same haplotype signature for this particular region of interest, sequences (e.g. sequencing reads) of these genotypes derived from the region of interest can all be treated as sequences of this haplotype group in the region of interest. Whereas individual genotypes may have shallow sequencing depths (e.g. 3×), the accumulation of all sequences for all genotypes within one haplotype group may reach a high enough depth (e.g. 100×) to achieve a reliable consensus sequence for this haplotype group that is more complete and of higher accuracy than the DNA sequence inferred from any single genotype. This haplotype consensus sequence can be generated by various methods including, but not limited to, assembly and sequence alignment according to the needs of the various consensus creation methodologies. The consensus sequence is referred to herein as an “Allele Model”.

In the example of an assembly-based consensus creation process, the sequencing depth of the haplotype group was calculated by adding up the various sequencing depths of all genotypes in the group. When the total sequencing depth of a haplotype group exceeded a minimum depth cutoff (e.g. 30×) for achieving reliable assemblies, local assembly was applied to the group.

For haplotype groups with enough sequencing depth, all or a subset selected by some criteria (e.g. mapping quality scores), of the sequences mapped to the region of interest were gathered and then fed into a public assembly tool (e.g. Pilon) to generate a consensus sequence.

The consensus sequence conveys the DNA sequence variants carried by the haplotype, and also identifies regions where the sequence of the haplotype group remains uncertain or unresolved. In a preferred method, a suitable spanning reference sequence is substituted for any unimproved or unresolved regions within the consensus.

### Sequence Assembly for Rare Haplotype Groups

Rare haplotype groups (those containing a small number of inbreds) may not contain sufficient sequence read coverage to enable a local assembly. To improve the sequence of such rare haplotypes, a preferred approach is to use a jumping profile hidden Markov model (HMM) to enable segmental alignment of the rare haplotype to the major haplotypes. Jumping profile HMMs (Schultz et al. 2006; Schultz et al. 2009) are an extension of profile HMMs to multiple profiles. In this approach, multiple alignments of inbred haplotypes or sequences representing each major haplotype group are used to create a HMM profile for each major haplotype. Given the suite of multiple profiles for a region of interest, a modified Viterbi algorithm (Schultz et al. 2006) may be used to determine the most likely path along the nucleotide sequence by which the rare haplotype could be produced by the major haplotype profiles. The resulting sequence segments map a rare haplotype to one or more major haplotypes, and switches in the aligned major haplotype profile are termed a breakpoint (FIG. 1). Rare haplotypes lacking evidence of breakpoints may be assigned to the most likely major haplotype group to which they are mapped. Rare haplotypes with identified breakpoints have subsequences flanking the breakpoint reassigned to the relevant major haplotypes. A number of other methods are available to identify potential breakpoints within sequences, examples include RDP (Martin and Rybicki 2000), Simplot (Lole et al. 1999), GENECONV (Sawyer 1989), et al.

### Edit Site Candidate Identification

A preferred approach for editing sequences is to use an editing compound which may be guided to edit a target sequence through provision of a guide nucleotide sequence with a degree similarity to the site to be edited. Editing systems that operate in this fashion include Cas9, Cpf1, C2c1 among others. Alternative editing compounds such as meganucleases, and TALENs among others, may recognize specific sets of sites, or those with a certain composition or characteristics. Characteristics of the ideal sites for modification vary in accordance with the requirements of the specific editing compounds. Site requirements may be applicable broadly to members of a given class or type of editing compound and the specific editing compound being used may have additional or modified requirements. For example, the single guide RNA (sgRNA) systems first described as the Type II CRISPR/Cas immune system of bacteria have been successfully repurposed as a genome engineering tool and the list of specific editing compounds of this type available to those skilled in the art of genome editing has continued to expand beyond those initial descriptions. Most members of this class share similar requirements for guide sequences within a preferred range of lengths, require presence of a protospacer-adjacent motif (PAM) near the modification location and require a degree of similarity to the guide for successful targeting. Specific parameters for length and motif and sequence content vary among editing compounds of this class but a number of guide RNA (gRNA) design tools have been developed recently that can accommodate them for this class of genome editing compound. Examples include Cas-OFFinder (Bae et al, 2014), GT-Scan (O'Brien et al, 2014), CCTop (Stemmer et al, 2015), CRISPRdirect (Naito et al, 2015), Off-Spotter (Pliatsika & Rigoutsos, 2015), CRISPRscan (Moreno-Mateos et al, 2015) and Breaking-Cas (Oliveros et al, 2016). Most of the tools identify potential gRNA targets by detection of user customizable PAM motif sequences and prediction of off-targets in whole genome sequences. Among them, a few tools support customizable maximum number of mismatches in off-targets (e.g. CRISPRdirect), or provide rankings to off-targets (e.g. Breaking-Cas). However, no tools provide the combination of customizable PAM motif sequences, customizable maximum number of mismatches, ranked off-targets and none of the tools provide the means to report specificity in sequence collections with non-native sequence abundances such as short read sequencing data with applicability to multiple types of genome editing compounds and systems. Described below are improved methods to identify preferred potential target sites for a given sequence or sequence region with a high probability for success.

### PAM Site Scan for CRISPR Associated Editing Compounds

Multiple approaches were used to locate editing sites among targeted sequence sets in the maize editing example conferring the waxy trait phenotype to specific maize genotypes using a preferred Cas9 editing compound. Targeted sequences were scanned to identify all PAM site locations on both strands. Targeted sequences may comprise limited regions within a set of sequences being analyzed, subset of sequences in the set, or include the entire sequence collection. Many methods for detection of a potential PAM site are available to a genome editing practitioner. In some approaches a window of the expected size of the PAM is searched for a match to the required nucleotides for that genome editing compound. In other cases, a statistical probability can be calculated for identification of sequence locations matching the PAM base probability profiles. Also a short window of length equal to the requirements of the PAM may be used to scan for matches along the length of sequences in the sequence set. In other methods sequences in the set to be queried can be broken into subsequences called kmers and these are used to identify possible PAM locations. Another example would be the use of dynamic programming alignment approaches to find sites. Yet another could rely upon use of alternative sequence set representations such as suffix arrays or sequence graph models to retrieve all sequences containing a match to the editing compound match requirement. There exists a vast array of software tools to detect complete or partial sequence matches to those skilled in the art.

For each PAM site, target sequences falling within the range of efficient recognition by the editing compound (e.g. 17nt to 25nt for Cas9) and in the proper relative positioning to the particular editing compounds needs relative to the detected PAM site were defined as candidate target sites. To illustrate with Cas9, the target sequence was defined as a gRNA sequence followed by the PAM sequence. For example, if the PAM is NGG, the target sequence is a 23nt sequence with a 20nt gRNA followed by a 3nt PAM. In a preferred embodiment an additional requirement is that the identified recognition sequence(s) start with a nucleotide G. These represent the pool of candidate editing sites from which the actual sites to edit were edited as described below.

### Candidate Identification for Other Classes of Editing Compounds

Candidate sites for editing compounds with sequence motif or composition-based restrictions on their sites of action may be identified using the same set of detection methods summarized for PAM site detection, simply suitably modified for the specific requirements of the given editing compounds.

For those editing compounds that require a certain sequence characteristic for site recognition, other detection approaches may be necessary. For example, if a certain structural conformation of the potential modification site is also needed, nucleotide structure prediction tools may be needed to delimit the location with potential for editing and then the sequences from those locations become the candidate pool.

### Physical Identification of Modifiable Sites

Sites suitable for editing may also be identified by a number of other means including but not limited to: in vitro or in vivo nucleotide protection assays and other methods to detect editing compound localizations on nucleotide sequences. For some detection methods, the editing compounds must be inactivated in order to retain the necessary localization. In other methods, suitable sites can be identified empirically through sequencing regions flanking sites of sequence modification. In other approaches if there is a nucleotide structural requirement, methods which enrich for sequences in the set with that structural class of motifs may be used to collect potential modification targets. For example, gel mobility assays may be performed on a sheared version of the targeted sequence set. In yet other approaches primers may be designed to known recognition motifs and used to amplify and or sequence all members in the target sequence set with primer binding. The collection of site sequences generated by any of these or other methods in common use by those skilled in the art become the candidate edit site sequence pool.

### Target Site Context (TSC)

It would be desirable to select the best sites to edit in terms of efficacy, specificity and efficiency of the desired modifications. Context information for editing sites can be provided in a number of ways to facilitate determination of which site(s) to use. A number of filters may be applied against members of the candidate pool to reduce the set of candidate sites for modification and apply prioritizations based upon how well they are expected to satisfy the desired qualities of specificity, modification efficiency, sensitivity, and ease of use.

For single guide RNA editing compounds a preferred requirement is that potential target sites start with a nucleotide G and end with the appropriate PAM for that editing compound to enable efficient U6 polIII guide sequence expression.

In general, site length filters may be applied to all types of genome modification agents during the design and creation of genome edited products guided by the recognition site needs of the sequence editing agent. For example, recognition sequence components of common Cas9 sites may be required to fall between 17nt and 25nt.

### Specificity Filters

Multiple approaches were used to determine specificity among sequence sets. The specific approach used depends upon whether the sequence set was expected to reflect the native abundance of the sequences. For example, reference genome sequences or other types of unamplified sequences may be used to reflect native abundance. Or if the modification sequence set contains potentially altered abundances, for example, PCR-amplified next generation sequence reads, then a corresponding altered sequence set may be used. These approaches apply to the maize editing example conferring the waxy trait phenotype to specific maize genotypes using a preferred Cas9 editing compound.

A filter often employed to improve specificity was to report only those sites with a unique or rare (default 2 instances) sequence and/or key sub-sequence(s) (e.g. the so called CRISPR/Cas9 seed sequence) in the collection of sequences being edited. Efficacy was also enhanced by filtering of candidate edit sites that have similar but not identical sequences or key sub-sequences in the sequence set with edit distances (default 4) within a range recognized by the pertinent editing compound. Presence of sites in the collection of sequences to be edited may be detected using short read aligners (e.g. Bowtie, BWA) or any of the other methods indicated in the PAM Selection section above or in common use by those skilled in the art. Edit distance was calculated for every detected hit by comparison of the hit sequence with that of the target site sequence. The calculation was performed as follows: each mismatch base has an edit distance of 1, each insertion or deletion has edit distance of its length. When there are ambiguity nucleotides (e.g. IUPAC codes) in either the target site sequence or the detected hit sequence, they were not penalized and are given an edit distance of 0.

In collections of sequences with potentially modified abundances, it is often useful to modify the candidate selection approach used to determine likely specificity within the set. The amount of data may impose additional challenges in determination of likely specificity of candidate modification sites. For example, if the target set exists as Illumina short read data, there may be hundreds of millions or even billions of reads. Additionally, sequence errors due to the sequencing platform or other causes may be present. Pre-processing of raw sequence data in these types of sequence sets, becomes necessary. In a preferred embodiment, pre-processing include steps to improve the reliability of the sequence. For example, trimming of adapter sequences, removal of PCR duplicates, overlapped sequence merging, sequence error correction, and collapse of identical sequences. These steps minimize the impact of ambiguity due to non-native abundances of sequences in the set to be modified on the detection of potential off-target hits. In our preferred embodiment, Cutadapt (Martin 2011) is used to trim adapter sequences, FLASH (Magoc and Salzberg 2011) is used to merging overlapping sequences, and BFC (Li 2015) is used for sequence error correction.

One method to reduce the impact of sequence set scale is to run steps which do not rely upon full knowledge of the sequence set simultaneously in parallel, on either the entire set or sub-sets of the starting sequence collection. Some steps such as a preferred method of sequence correction require access to the entire dataset and thus cannot be chunked and must be run in a sequential manner.

Alternatively, many of these steps can be replaced or superseded through use of specialized methods of organizing sequence data such as the aforementioned sequence graph models, some forms of which will inherently reduce redundant information in the dataset and improve reliability of sequences.

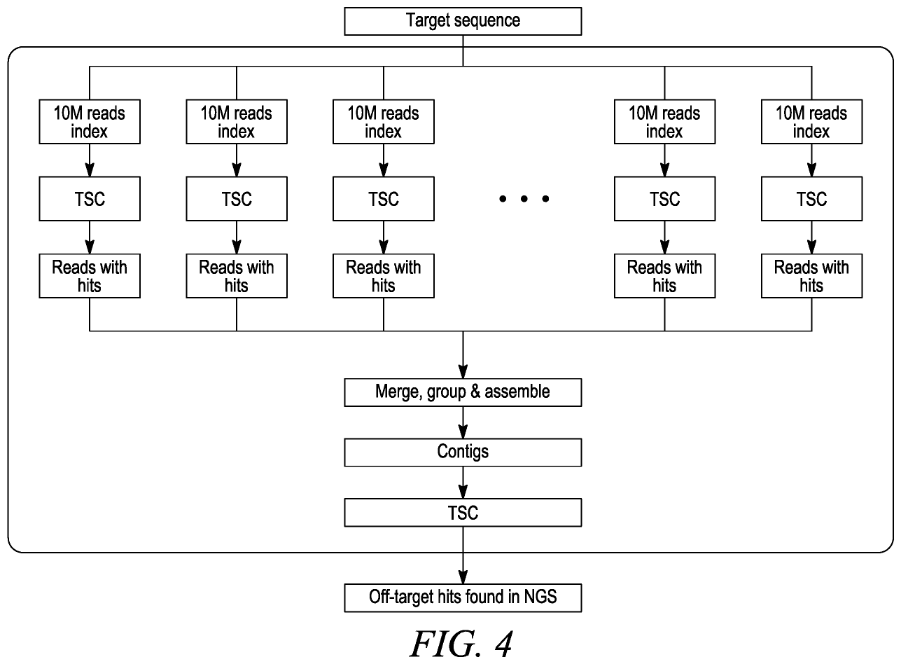

After sequence set consolidation and clean up, the modified dataset used to find target sequences is searched for sequences with similarity to members of the candidate site pool to create a set of detected potential sites as previously described for native abundance sequence sets. In a preferred embodiment, sequences in the cleaned target sequence set with a detected site are grouped by the matched candidate pool site. Assembly is applied within each group to reduce the possibility of mis-assembly and to generate a consensus context for the site, for example using CAP3 (Huang and Madan 1999). Sequences in each group are then assembled into contigs to maximize the uniqueness of off-targets. Each contig represents an off-target locus in genome. Similarity cutoffs (for example, default 99% identity) are used to reduce the potential for over-collapse of sequences which are similar but derived from different sources. A second round of the selection process is then performed using the assembled contigs as the sequence set targeted for modification. FIG. 4 illustrates the process of specificity screening in non-native sequence abundance collections. The number of reads used in assembly and the number of ambiguity bases in the contigs are used as additional filtration factors in scoring each off-target locus.

### Additional Filters.

In the case of editing compounds with a PAM, the similar sequence must also satisfy the PAM requirements for that editing compound, including any alternative PAM sequence motifs (e.g. NAG for NGG for the originally described Streptococcus pyogenes (Spy) Cas9).

In a preferred embodiment, for each potential editing site, a number of features of the site sequence and its genomic context are reported. Examples of these include whether the site has 3+ consecutive Ts, Gs or Cs to assess potential for premature termination, potential for disruption of other features at that location (for example, genes or other annotation features), repetitive nature of the surrounding DNA, DNA methylation status, and whether the target site sequence is conserved in the genotypes to be edited if deep sequencing data is available. Many other characteristics of the site sequences or their surrounding context in the collection of sequences to edit will be available to those skilled in the art.

### Candidate Site Scoring

Weights are assigned to the status of each filter result for a site and a penalty score provided to simplify assessment of the potential for the desired modification to be made exactly as desired. In a preferred embodiment, the penalty weighting scheme is as follows:
Edit distance. The closer the edits, if any, are to the most constrained portions of a site (e.g. PAM sequences) the higher the penalty.
   Insertions and deletions have an extra penalty applied
Sites which include alternative, less preferred portions of the recognized region for an editing compound (e.g. secondary or alternative PAMs for single RNA guide editing compounds) are penalized.

### Example 1

A total of 12 inbred lines were selected as the target lines for Waxy genome editing. (See publication number PCT/US 17/14903, incorporated herein by reference, for details about the Waxy edited target lines). The proprietary Allele Model sequence repository includes Next Generation Sequencing (NGS) sequences for a total of 582 maize inbreds, 38 of them having relatively deep coverage (30×) with the remainder having an average of 3× coverage. All sequences were aligned to the B73 reference genome using Bowtie2 (Langmead et al. 2012). SNP loci were defined from the inbreds with relatively deep coverage. To be defined as a SNP, a locus must meet the following criteria:
1. At least one inbred displays a homozygous genotype that differs from the reference.
2. Only 4 inbreds (approximately 10% of the 38) are permitted to have missing data
3. Only 6% of inbreds with observed data may carry a heterozygous genotype. (In the case of all 38 inbreds showing observed data, this criterion would allow 2 inbreds to be heterozygous).
4. Only two homozygous alleles are observed for the locus across all inbreds.

A ‘homozygous’ genotype was defined as the case where at least 98% of the observed reads contain the same allele.

The genomic region of interest contained 66 SNP loci that were used to identify which inbreds are identical-in-state within the Wx gene region. The 66 locus genotypes of 582 inbreds yields a matrix of 38,412 possible genotype scores, of which 9,411 were unobserved. To facilitate haplotype construction in a high-throughput pipeline, these unobserved genotypes were imputed by a nearest-neighbor approach. Given an inbred of interest and a locus with an unobserved score, the genotypes of the 300 SNP loci surrounding that locus were compared to the genotypes of each other inbred in the dataset. The nearest-neighbor inbred was defined as the inbred with the lowest mismatch score relative to the inbred of interest at the SNP loci within the window of 300 SNPs. A mismatch score for a pair of inbreds consisted of a sum of the mismatch scores from each SNP locus in the genomic window (similar to Roberts et al. 2007). A mismatch between two homozygous genotypes was recorded as a score of 2, and sites with missing data were scored as 1. A mismatch in which one inbred was homozygous and the other heterozygous was also scored as 1. If more conservative imputation is desired, the mismatch scores of either missing data or heterozygous loci can be modified.

Inbreds were grouped into sets with haplotypes identical-in-state based on the similarity of the observed and imputed SNP genotypes across the 300 loci. The genotypes of all inbreds were assigned by choosing one of the two homozygous alleles at each locus to serve as an arbitrary reference allele. Genotypes that did not match the reference allele were recoded as 0, and genotypes that matched the reference allele were coded as 1. A missing genotype was recoded as 0.5. With the genotypes recoded into numeric values, the distance d between two inbreds was calculated from their genotypes as follows:

$$d ( a , b ) = {\sum }_{i = 1}^{n} \vert {a}_{i} - {b}_{i} \vert \qquad ( 1 )$$

where a and b are the vector of recoded genotypes for each inbred, and n is the number of SNP genotypes in the region of interest. This distance metric is commonly referred to as “Manhattan” distance. The inbreds were then clustered based on these distances in a hierarchical, agglomerative fashion using complete linkage, which is a standard approach to clustering problems (James et al. 2013). All inbreds were placed into their own cluster in the initial iteration. In successive iterations, all pairs of clusters were compared and the clusters with the smallest distance between them were joined. With the complete linkage method, the distance D between two clusters A and B is defined as:

$$D ( A , B ) = {max}_{a ∈ A , b ∈ B} d ( a , b ) \qquad ( 2 )$$

where d(a,b) is defined as in equation 1. A threshold t was chosen as the maximum allowable distance at which two clusters can be joined. Haplotypes groups were thus defined by the condition in which all pairs of clusters have distances greater than the threshold t:

∀A≠B:D(A,B)>t (3)

The use of Manhattan distance to define genotype distances and complete linkage to define cluster distances allows a haplotype group to be interpreted as consisting of the set of inbreds whose genotype distances were all less than the threshold t. The related value s defined as:

$$s = \frac{1 - t}{n} \qquad ( 4 )$$

can be thought of as a “similarity cutoff” that sets the minimum genotype similarity allowed within a haplotype group.

Execution of the aforementioned procedure of haplotype group assignment on the 582 inbreds with a similarity cutoff s=0.98 yielded 10 identical-in-state groups of at least 3 inbreds for the Wx region of interest.

### Example 2

This example demonstrates the use of nucleic acid targeting sequences designed in accordance with the methods of the invention to generate targeted genome edits while minimizing unintended off-target edits.

When guideRNA scenarios for Waxy1 (Wx1: GRMZM2G024993) were evaluated, candidate Cas9 target sites were identified in Allele Model sequences, followed by researcher's selection of target sites from the candidate pool, and then the selected targets were checked against the B73 reference genome and the allele model for the edited genotype or off-target sites.

A number of scenarios were explored for guideRNA design. In one embodiment, individual allele model sequences can be supplied to a web or command line interface implementing these methods, and output specific to each input Allele Model can be generated. Filtering preferences can be selected, for example minimization of off-target hits found in the Reference Genome(s), and the results compared to identify conserved nucleic acid targeting sequences.

Other embodiments include an examination of consensus sequences for the top ranking Allele Model sequences. In such embodiments, any acceptable Multiple Sequence Alignment (MSA) tool (for example, www.ebi.ac.uk/tools/msa) can be deployed to generate a consensus input sequence for examination via methods described in the Edit Site Candidate Identification section. ClustalW(2), MAFFT, MUSCLE, KALIGN or alternative programs available to one skilled in the art can be used to produce effective multiple sequence alignments and resultant consensus sequence assemblies. Programs such as Sequencher, AlignX, or other DNA/RNA/Protein sequence software suites often contain embedded ClustalW or other MSA tools and can output consensus sequences in various formats such as FASTA. Consensus files can be generated using default or custom parameters controlling how the consensus is derived (identity/plurality) and how nucleotide or residue polymorphisms can be displayed using IUPAC codes for polymorphic nucleotides. In a preferred embodiment, a consensus sequence file, produced by aligning more than two allele model groups, was submitted to command line or web tools encapsulating the methods described above to search for suitable sites which, when selected for design of guideRNAs, enabled Cas9 editing compounds to make edits to all major haplotype groups in the Waxy1 Allele Model with the same editing compound. Consensus sequences and multiple alignments of haplotypes were used to identify suitable sub-regions of the Waxy1 allele model with a high degree of sequence similarity so that multiple haplotypes may be efficiently targeted by the same editing compound. Additionally, consensus sequences and alignments of haplotypes for the targeted region were used to identify locations which, if targeted by an editing compound capable of targeting that site, would direct it to modify only certain haplotypes or groupings of haplotypes which share targetable sequence conservation among themselves but differ materially from other haplotypes at that site. Any IUPAC substitution residues were converted to the any-base code N by web site and command line tools implementing the methods described in the Edit Site Candidate Identification and Selection Among Edit Site Candidates sections when searching for off-site hits.

In a preferred embodiment, consensus files generated via MSA Tools can be subjected to any of the numerous bioinformatic repeat masking algorithms known to practitioners of genome editing, which filter out sequence repetitive residues based on their similarity relationships to sequences known or discovered to be repetitive for any genome, or for interspersed repeats identified de-novo using a multitude of approaches accepted in the art. In a preferred embodiment, a consensus allele model sequence derived from any MSA tool can be submitted, with or without IUPAC substitutions for polymorphic residues, to repeat masking algorithms that produce output files which mask repetitive residues with ambiguous placeholders such as X or N.

Example (double-stranded) Repeat-masked Waxy1 (promoter) consensus Allele Model sequence, indicating conserved guideRNA targets CR10 and CR4.

| | |
|---|---|
| 1 | TAGCTACGTG CCTGCTCATG ATCAGAACCC CAGACCACGA TCTGCGTGCT |
| | ATCGATGCAC GGACGAGTAC TAGTCTTGGG GTCTGGTGCT AGACGCACGA |
| 51 | AGCTTCCTCT TGCACTGGCG ATCCCGTCGT GTCGTCTCTG CCTCTNNNNN |
| | TCGAAGGAGA ACGTGACCGC TAGGGCAGCA CAGCAGAGAC GGAGANNNNN |
| 101 | NNNNNNNNNN NNNNNNNNAC TTGNCACNGC ATGCNACTCC ATTGCGAGNG |
| | NNNNNNNNNN NNNNNNNNTG AACNGTGNCG TACGNTGAGG TAACGCTCNC |
| 151 | GGNAGAAGAA AAGGGNGAGA AGACCAGAGG GAAAAACACT ACGCGCCTAT |
| | CCNTCTTCTT TTCCCNCTCT TCTGGTCTCC CTTTTTGTGA TGCGCGGATA |
| 201 | ATATGNNNNN NNNNNNNNNN NNNNNNNNNA GCTAGNNNNN NNNNNNNNNN |
| | TATACNNNNN NNNNNNNNNN NNNNNNNNNT CGATCNNNNN NNNNNNNNNN |
| 251 | NCCGCAGCTT NNANNCNNNN AGCTTAANAA CATTGGNTAA NTAATAATNA |
| | NGGCGTCGAA NNTNNGNNNN TCGAATTNTT GTAACCNATT NATTATTANT |
| 301 | TCGTAACCTC TTGTACGTCC CGACTAGCTA GTCTACCAAC CCACCCACGC |
| | AGCATTGGAG AACATGCAGG GCTGATCGAT CAGATGGTTG GGTGGGTGCG |
| 351 | TGAGCTTTCA ATCGCNCAAG GAGAAAGAAT AATCGAGANG ACGGCACAGG |
| | ACTCGAAAGT TAGCGNGTTC CTCTTTCTTA TTAGCTCTNC TGCCGTGTCC |
| 401 | ANAGCTAAAA CAAAAGCCTT GTAGTTATGG ATGAAGANGA AGATGATGAT |
| | TNTCGATTTT GTTTTCGGAA CATCAATACC TACTTCTNCT TCTACTACTA |
| 451 | AACACANAAT ATTTAAGTTT GGTNTGTGTG GCTAAGCAGT GGAAACACAC |
| | TTGTGTNTTA TAAATTCAAA CCANACACAC CGATTCGTCA CCTTTGTGTG |
| 501 | ACNCANNCNN ANGCATANAN AGAAAAACAA TGAAACTTTA AACTAGAACG |
| | TGNGTNNGNN TNCGTATNTN TCTTTTTGTT ACTTTGAAAT TTGATCTTGC |
| 551 | ACAAGAAGAC GAGAGCTAAT ATTATGGAAG GGTCTTGATA TTNCNCNNGA |
| | TGTTCTTCTG CTCTCGATTA TAATACCTTC CCAGAACTAT AANGNGNNCT |
| 601 | ANNANGCTNC ACGAACTACA CAANAAANNN NNNNNNNNNN NNNATANTTA |
| | TNNTNCGANG TGCTTGATGT GTTNTTTNNN NNNNNNNNNN NNNTATNAAT |
| 651 | AGGTTGGCTT TTNNAAAAGG GCATGTGAAA AAAAAAGGTA GAACGGNNNN |
| | TCCAACCGAA AANNTTTTCC CGTACACTTT TTTTTTCCAT CTTGCCNNNN |
| 701 | NNNNNNNNNN NNNATCAGAT CGATGCTCTG CATATGGAGA TCAGGTTAAG |
| | NNNNNNNNNN NNNTAGTCTA GCTACGAGAC GTATACCTCT AGTCCAATTC |
| 751 | ACAGCAATTA ATTTGATGCC GTCCTATNTA TCGGAAAACN TGTCAAAGNG |
| | WX1_PRO_CR10 |
| | ~ |
| | TGTCGTTAAT TAAACTACGG CAGGATANAT AGCCTTTTGN ACAGTTTCNC |
| 801 | CTGGGAGAGA CGGTGTAGTA GGGGGGCATC NAAACATTCA CACTAAAATG |
| | PAM (GGG) |
| | ~~~ |
| | WX1_PRO_CR10 |
| | ~~~~~~~~~~~~~~~~~~~~~ |
| | GACCCTCTCT GCCACATCAT CCCCCCGTAG NTTTGTAAGT GTGATTTTAC |
| 851 | GTGCCATGTA GGACACTACT TCNNNNNNNN NNNNNNNNNN NNNNGAGTTG |
| | CACGGTACAT CCTGTGATGA AGNNNNNNNN NNNNNNNNNN NNNNCTCAAC |
| 901 | GGAGAGTTTT TTCGGTACAN NNNNNNNNNN NNNNNCTCCA CTCTAGGCTT |
| | CCTCTCAAAA AAGCCATGTN NNNNNNNNNN NNNNNGAGGT GAGATCCGAA |
| 951 | CCCACAGTGG GCCAGACACC TTGGCGCTAG GCTTGACGAT CCTCTTGGGC |
| | GGGTGTCACC CGGTCTGTGG AACCGCGATC CGAACTGCTA GGAGAACCCG |
| 1001 | CTACTGTTGG GCTTGTGTCG CTGGTCACGC GGGCCTTGTG GCACACATTG |
| | GATGACAACC CGAACACAGC GACCAGTGCG CCCGGAACAC CGTGTGTAAC |
| 1051 | GGATGACTGG CACTCTCTTC CTCGTTGGGC TTGCGGAAAC TGTTGGCGCA |
| | CCTACTGACC GTGAGAGAAG GAGCAACCCG AACGCCTTTG ACAACCGCGT |
| 1101 | AGCAAAAGGC TTTGAGACTT CGCAGGTAGC CGAGTGTTGC TTGCTGGCAT |
| | TCGTTTTCCG AAACTCTGAA GCGTCCATCG GCTCACAACG AACGACCGTA |
| 1151 | GTGTGATGTG ATTCCNNNNN NNNNNNNNNN NNNNNNNNNN NNNNNNNACG |
| | CACACTACAC TAAGGNNNNN NNNNNNNNNN NNNNNNNNNN NNNNNNNTGC |
| 1201 | GGTGACCAAT ACTAACATCG TATTGTACCT GCTCGACAAC TATNNGAAGA |
| | CCACTGGTTA TGATTGTAGC ATAACATGGA CGAGCTGTTG ATANNCTTCT |
| 1251 | CATTNNAANT NANANNNNGA NNNNNNNNNN NANNGANNNT ACTACATCGG |
| | GTAANNTTNA NTNTNNNNCT NNNNNNNNNN NTNNCTNNNA TGATGTAGCC |
| 1301 | AGTTCANAAA CAATTGATGT ATGCTCCTCG GATTGCCACA GTGNGCCGAA |
| | TCAAGTNTTT GTTAACTACA TACGAGGAGC CTAACGGTGT CACNCGGCTT |
| 1351 | TACTTTGGCA CTANGCTTCA CGGTGCTCCT GGGCCTAGCG TTCAGAGTGA |
| | ATGAAACCGT GATNCGAAGT GCCACGAGGA CCCGGATCGC AAGTCTCACT |
| 1401 | GCTCTCGCTT CCAATGTTGG GCCTGTGNNN NNNNNNNNNN NNNNNTCAGA |
| | CGAGAGCGAA GGTTACAACC CGGACACNNN NNNNNNNNNN NNNNNAGTCT |
| 1451 | TTGGCTAAGN CTATNTTCGG NTGNTTANCT ATCTCNGTAT NTATATTNAA |
| | AACCGATTCN GATANAAGCC NACNAATNGA TAGAGNCATA NATATAANTT |
| 1501 | ACTCCACTCT ANAAANTATA GTATAATATA GTGATTTGAN TGACTATATG |
| | TGAGGTGAGA TNTTTNATAT CATATTATAT CACTAAACTN ACTGATATAC |
| 1551 | NGTGNACTGC TNGAGACGAC CTAACCATGA GGAAAGAAAN ACTTTGAACA |
| | NCACNTGACG ANCTCTGCTG GATTGGTACT CCTTTCTTTN TGAAACTTGT |
| 1601 | TCAAGNAGNN NNNNNNNNNN NNNNNNTCGA TACGTAATAA CGTGTGTACG |
| | AGTTCNTCNN NNNNNNNNNN NNNNNNAGCT ATGCATTATT GCACACATGC |
| 1651 | CNNGTANANA ATAACCAAAA TATNTTAGAA TGCATCTAGT TAATNAAATT |
| | GNNCATNTNT TATTGGTTTT ATANAATCTT ACGTAGATCA ATTANTTTAA |
| 1701 | AGGTTCTTTG AGCCTAANCA CTGANNNTAA GCANTTTGTT TCTAGACCAA |
| | TCCAAGAAAC TCGGATTNGT GACTNNNATT CGTNAAACAA AGATCTGGTT |
| 1751 | ATTTCATGGT AGTTGGGAGC CTACCCANAT TTCANNATTA ANTGTGCTAT |
| | TAAAGTACCA TCAACCCTCG GATGGGTNTA AAGTNNTAAT TNACACGATA |
| 1801 | TGAATTGNTG AAAATGNNTG TGTNTGTCNT ATNCGACGGA TAACGNNNNN |
| | ACTTAACNAC TTTTACNNAC ACANACAGNA TANGCTGCCT ATTGCNNNNN |
| 1851 | NNNNNNNNNN NTCNATGGGC ATGNGCATNG ATATAGATNT GTACCCACTA |
| | NNNNNNNNNN NAGNTACCCG TACNCGTANC TATATCTANA CATGGGTGAT |
| 1901 | CTAGTATGGT CGCAGNCGGA TATTGNTTGC AACCNCAGAT ATAGTTTCNG |
| | GATCATACCA GCGTCNGCCT ATAACNAACG TTGGNGTCTA TATCAAAGNC |
| 1951 | GGAAAAGGAT TAGGCTCAGC TCCATCCCTA GACCCCANTN GNNNNNNNNN |
| | CCTTTTCCTA ATCCGAGTCG AGGTAGGGAT CTGGGGTNAN CNNNNNNNNN |
| 2001 | GNGNGNGGGG GTCTACCCTT CAAAANGAAA AAAAACTACA CACAGTGCAT |
| | CNCNCNCCCC CAGATGGGAA GTTTTNCTTT TTTTTGATGT GTGTCACGTA |
| 2051 | ATAAGAAGAT GAATATTCCA AAATTCAGCA GTCAAGAAGC CCTGATAAAC |
| | TATTCTTCTA CTTATAAGGT TTTAAGTCGT CAGTTCTTCG GGACTATTTG |
| 2101 | TGTCTGGCAT AGCTAGTACT TTATACACTT CAAGACCAAA AGAAATCACT |
| | ACAGACCGTA TCGATCATGA AATATGTGAA GTTCTGGTTT TCTTTAGTGA |
| 2151 | AAGTACAGAT TTTAGTGACT CGTAAGTACA GATATCATCT TACAAGGCCC |
| | TTCATGTCTA AAATCACTGA GCATTCATGT CTATAGTAGA ATGTTCCGGG |
| 2201 | AGCCCAGCGA CCTATTACAC AGCCNNNNNN NNNNNNNNNN NTCGGGACAC |
| | TCGGGTCGCT GGATAATGTG TCGGNNNNNN NNNNNNNNNN NAGCCCTGTG |
| 2251 | ANNNNNNNNN NNNNNNNNGT GAAGCTCTGC TCGCAGCTGT CCGGCTNCTT |
| | TNNNNNNNNN NNNNNNNNCA CTTCGAGACG AGCGTCGACA GGCCGANGAA |
| 2301 | GGACGTTCGT GTGGCAGATT CATCTGTNGT CTCGTCTCCT GTGCTTCCTG |
| | CCTGCAAGCA CACCGTCTAA GTAGACANCA GAGCAGAGGA CACGAAGGAC |
| 2351 | GGTAGCTTGT GNAGTGGAGC TGACATGGTC TGAGCAGGCT TAAANNTTNN |
| | CCATCGAACA CNTCACCTCG ACTGTACCAG ACTCGTCCGA ATTTNNAANN |
| 2401 | NNNNNNNNNN NNNNNNNNNN NNNNNNNNNN NNNNNNNNNN NNNNNNNNNN |
| | NNNNNNNNNN NNNNNNNNNN NNNNNNNNNN NNNNNNNNNN NNNNNNNNNN |
| 2451 | NNNNNNNNNN NNNNNNNNNN NNNNNNNNNN NNNNNNNNNN NNNNNNNNNN |
| | NNNNNNNNNN NNNNNNNNNN NNNNNNNNNN NNNNNNNNNN NNNNNNNNNN |
| 2501 | NNNNNNNNNN NNNATNNNNN NNNNNNNNNN NNNNNNNNNN NNNNNNNNNN |
| | NNNNNNNNNN NNNTANNNNN NNNNNNNNNN NNNNNNNNNN NNNNNNNNNN |
| 2551 | NNNNNNNNNN NNNNNNNNNN NNNNNNNNNN NNNNNNNNNN NNNNNNNNNN |
| | NNNNNNNNNN NNNNNNNNNN NNNNNNNNNN NNNNNNNNNN NNNNNNNNNN |
| 2601 | NNNNNNNNNN NNNNNNNNNN NNNNNNNNNN NNNNNNNNNN NNNNNNNNNN |
| | NNNNNNNNNN NNNNNNNNNN NNNNNNNNNN NNNNNNNNNN NNNNNNNNNN |
| 2651 | NNNNNNNNNN NNNNNNNNNN NNNNNNNNNN NNNNNNNNNN NNNNNNNNNN |
| | NNNNNNNNNN NNNNNNNNNN NNNNNNNNNN NNNNNNNNNN NNNNNNNNNN |
| 2701 | NNNNNNNNCT GAGCAGGNNN AAAATTTGCT CGTAGACGAG GAGTACCAGC |
| | NNNNNNNNGA CTCGTCCNNN TTTTAAACGA GCATCTGCTC CTCATGGTCG |
| 2751 | ACAGCACGTT GCGGATTTCT CTGCCTGTGA AGTGCAACGT CTAGGATTGT |
| | TGTCGTGCAA CGCCTAAAGA GACGGACACT TCACGTTGCA GATCCTAACA |
| 2801 | CACACGCCTT GTGGTCGCGT CGCGTCGATG CGGTGGTGAG CAGAGCAGCA |
| | GTGTGCGGAA CACCAGCGCA GCGCAGCTAC GCCACCACTC GTCTCGTCGT |
| 2851 | ACAGCTGGGC GGCCCAANGT TGGCTTCCGT GTCTTCGTNN NNNNNNNNNN |
| | TGTCGACCCG CCGGGTTNCA ACCGAAGGCA CAGAAGCANN NNNNNNNNNN |
| 2901 | NNNNNNNNNN NNNNNNAGCA GAGAGCGGAG ANCGAGCCGT GCACGGGGGA |
| | NNNNNNNNNN NNNNNNTCGT CTCTCGCCTC TNGCTCGGCA CGTGCCCCCT |
| 2951 | GGTGGTGTGN AAGTGNANNN NNNNNNNNNN NNNNNNNNNN NNNNTGGGCA |
| | CCACCACACN TTCACNTNNN NNNNNNNNNN NNNNNNNNNN NNNNACCCGT |
| 3001 | ACCCAAAAGT ACCCACGACA AGCGAAGGCG CCAAAGCGAT CCAAGCTCCG |
| | TGGGTTTTCA TGGGTGCTGT TCGCTTCCGC GGTTTCGCTA GGTTCGAGGC |
| 3051 | GAACGCANCA GCNNAGCNTC GCGTCGNNNN GGAGNGCANC AGCCACAAGC |
| | CTTGCGTNGT CGNNTCGNAG CGCAGCNNNN CCTCNCGTNG TCGGTGTTCG |
| 3101 | AGCCGAGAAC CGAACCGGTG GGCGACGCGT CNTGGGACGG ACGCGGGCGA |
| | TCGGCTCTTG GCTTGGCCAC CCGCTGCGCA GNACCCTGCC TGCGCCCGCT |
| 3151 | CGCTTCCAAA CGGGGCCACG TACGCCGNNN NNNNNNNNNN NNNNNNNNNA |
| | GCGAAGGTTT GCCCCGGTGC ATGCGGCNNN NNNNNNNNNN NNNNNNNNNT |
| 3201 | CGACAAGCCA AGGCGAGGCA GCCCCCGATC GGGAAAGCGT TTTGGGCNNN |
| | GCTGTTCGGT TCCGCTCCGT CGGGGGCTAG CCCTTTCGCA AAACCCGNNN |
| | ~~~ |
| | PAM (CGG) |
| | ~~~~~~~~~~~~~~~~~~~~~~ |
| | CR4 |
| 3251 | NNNNNNNGCG TGCGGGTCAG TCGCTGGTGC GCAGTGCCGG GGGGAACGGG |
| | NNNNNNNCGC ACGCCCAGTC AGCGACCACG CGTCACGGCC CCCCTTGCCC |
| 3301 | TATCGTGGGG GGCNNNNNNN NNNNNNNNNG TGGCGAGGGC CGAGAGCAGC |
| | ATAGCACCCC CCGNNNNNNN NNNNNNNNNC ACCGCTCCCG GCTCTCGTCG |
| 3351 | GCGCGGCCGG GTCACGCAAC GCGCCCCACG TACTGCCCTC CCCCTCCGCG |
| | CGCGCCGGCC CAGTGCGTTG CGCGGGGTGC ATGACGGGAG GGGGAGGCGC |
| 3401 | CGCGCTAGAA ATACCGAGGC CTGGACCGGG GGNNGCCCCN NCNCNGTCAC |
| | GCGCGATCTT TATGGCTCCG GACCTGGCCC CCNNCGGGGN NGNGNCAGTG |
| 3451 | ATCCATCNAN CGANCGATCG ATCGCCACAG CCAACACCAC CCGCCGAGGC |
| | TAGGTAGNTN GCTNGCTAGC TAGCGGTGTC GGTTGTGGTG GGCGGCTCCG |
| 3501 | GACGCGACAG CCGCCNNNNN NNNNNNNNNN NCTCACTGCC AGCCAGTGAA |
| | CTGCGCTGTC GGCGGNNNNN NNNNNNNNNN NGAGTGACGG TCGGTCACTT |
| 3551 | GGGGGAGAAG TGTACTGCTC CGTCNACCAG TGCGCGCACC GCCCGGCAGG |
| | CCCCCTCTTC ACATGACGAG GCAGNTGGTC ACGCGCGTGG CGGGCCGTCC |
| 3601 | GCTGCTCATC TCGTCGACGA CCAG (SEQ ID NO: 1) |
| | CGACGAGTAG AGCAGCTGCT GGTC (SEQ ID NO: 2) |

### Example 3

The repeat-masked Waxy1 consensus Allele Model sequence was run through a PAM site scan to identify all PAM sites and then filtered to those candidates that have no more than a single copy of the exactly matched target sequence in the reference genome sequence. Bowtie (“bowtie-a-v0”) was used to search for exact match hits of target sequences in a maize reference genome. In total, 109 target PAM sites were identified with at most one copy of an exact target sequence, and among them, there were 68 target PAM sites with at most one copy of the seed sequence, which became the candidates.

Next, the target sequence of each candidate PAM site was run through reference-based off-targets scan to identify all possible off-targets with up to 4 edit distance using BWA (“bwa aln-n 4”). The off-targets that were not exactly identical but very similar to the target sequence were found in the reference genome and then used to further filter the candidate list to those with no 1-edit distance off-targets. For example, the number of off-targets with 0 to 4 edit distances in the Maize B73 reference genome were listed for CR4 and CR10. There were off-targets with edit distances greater than 2 for both sites but the total number was low enough to confirm both sites were specific to the waxy sequence.

Lastly, each target sequence was run through the reference-free off-targets scan to identify all possible off-targets with edit distances up to 4 in the NGS short reads of three maize inbred lines, where each inbred line had been sequenced at 75×+depth using Illumina Hi-Seq. The off-targets found in the NGS reads were then further confirmed that no exact match hits in these inbreds were found other than the target sequence. For example, the number of off-targets with 0 to 4 edit distances in inbreds for CR4 and CR10 were listed below. Two contigs were found with exact matches in INBRED2_NGS for CR10 but then the two contigs were confirmed as coming from same source by another round of assembly using CAP3 where identity cutoff was relaxed to 95%. The same applied to the two contigs with exact match in INBRED1_NGS for CR4. The number of off-targets at each edit distance were still low enough to confirm their specificity to the waxy sequence.

TABLE 1

| | | | | NUMBER OF OFF-TARGETS | | | | |
|---|---|---|---|---|---|---|---|---|
| REF_NAME | ID | TARGET_SEQ | PAM | ED = 0 | ED = 1 | ED = 2 | ED = 3 | ED = 4 |
| MAIZE_B73 | CR10 | GCTGGGAGAGACGGTGTAGTAGGG | GGG | 1 | 0 | 1 | 4 | 8 |
| INBRED1_NGS | CR10 | GCTGGGAGAGACGGTGTAGTAGGG | GGG | 1 | 2 | 0 | 12 | 65 |
| INBRED2_NGS | CR10 | GCTGGGAGAGACGGTGTAGTAGGG | GGG | 2 | 1 | 0 | 12 | 95 |
| INBRED3_NGS | CR10 | GCTGGGAGAGACGGTGTAGTAGGG | GGG | 1 | 1 | 1 | 12 | 91 |
| | | | | NUMBER OF OFF-TARGETS | | | | |
| REF_NAME | ID | TARGET_SEQ | PAM | ED = 0 | ED = 1 | ED = 2 | ED = 3 | ED = 4 |
| MAIZE_B73 | CR4 | GCCCAAAACGCTTTCCCGATCGG | CGG | 1 | 0 | 0 | 6 | 13 |
| INBRED1_NGS | CR4 | GCCCAAAACGCTTTCCCGATCGG | CGG | 2 | 0 | 1 | 11 | 50 |
| INBRED2_NGS | CR4 | GCCCAAAACGCTTTCCCGATCGG | CGG | 1 | 0 | 0 | 20 | 50 |
| INBRED3_NGS | CR4 | GCCCAAAACGCTTTCCCGATCGG | CGG | 1 | 0 | 2 | 18 | 45 |

### Example 4

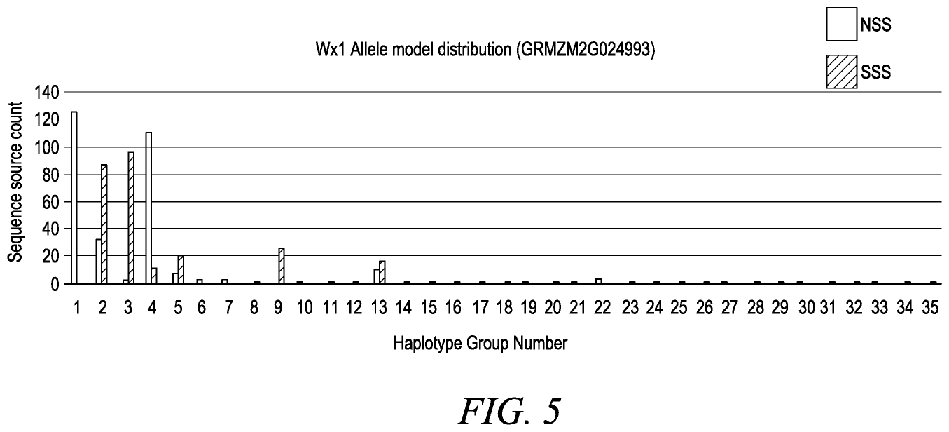

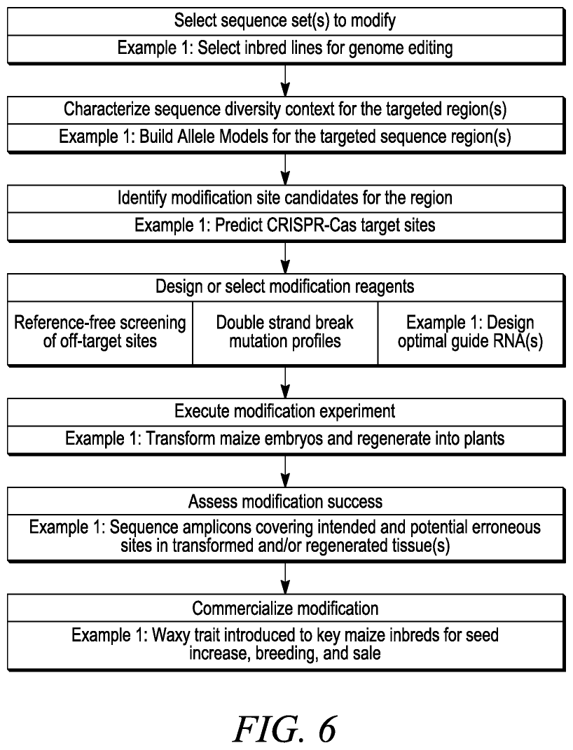

The overall distribution of haplotype groups can be examined with respect to typical heterotic groups contained within the cohort of 582 inbreds, such as Stiff Stalk Synthetic (SSS), Non-Stiff Stalk (NSS), Flint, or other heterotic group classifications. In the case of the Waxy1 gene (Wx1, GRMZM2G024993), the 10 identical-in-state groups can be parsed further into major Pilon assembly-based allele model groups within the SSS and NSS heterotic pools (see FIG. 5)

TABLE 2

| Pilon Group Number | NSS | SSS | Totals | Allele % | |
|---|---|---|---|---|---|
| 1 | 126 | | 126 | 21.65 | |
| 4 | 111 | 12 | 123 | 21.13 | |
| 2 | 32 | 87 | 119 | 20.45 | |
| 3 | 3 | 96 | 99 | 17.01 | |
| 5 | 7 | 20 | 27 | 4.64 | |
| 9 | | 26 | 26 | 4.47 | |
| 13 | 10 | 16 | 26 | 4.47 | |
| 22 | 4 | | 4 | 0.69 | |
| 6 | 3 | | 3 | 0.52 | |
| 7 | 3 | | 3 | 0.52 | 0.96 |

In this Wx1 example, the top 10 unique allele models represent 96% of all lines in the n=582 inbred set. Design of CRISPR-Cas experiments for Wx1 can be focused on individual allele models corresponding to a specific targeted inbred genotype, or focused on the predominant alleles observed in the allele model distribution, or focused on rare alleles from the allele model distribution, or focused on consensus sequence files generated by comparing two or more sequences from the allele model distribution. The guideRNAs described in SEQID No. 1, WX1_PRO_CR10, and WX1_PRO_CR4 as examples are 100% conserved across all major haplotypes, have minimum off-site targets detected by our web-based and command line-based implementation(s) of the site identification and selection methods reported above, and were expected to have activity as Cas9 reagents in cutting DNA across all major IIS haplotypes in relevant germplasm.

That which is claimed:

1. A method of designing a guide polynucleotide that minimizes the potential of generating off-target site gene edits, the method comprising:
a) comparing a target site sequence for an endonuclease against unassembled raw nucleotide sequence reads from individuals in a population;
b) assembling the raw nucleotide sequence reads that align with part or all of the target site sequence into individual contigs;
c) selecting the target site sequence comprising a single copy of the target sequence in the contigs from step b;
d) designing a guide RNA for that target site sequence; and
e) generating an intended gene edit at the target site in a nucleic acid using the designed guide polynucleotide in an endonuclease complex.

2. The method of claim 1, wherein the raw read nucleotide sequences are short or long read nucleotide sequence reads.

3. The method of claim 1, wherein the comparing comprises aligning the target sequence with the sequence from unassembled raw nucleotide sequence reads.

4. The method of claim 1, further comprising identifying whether the contig comprise two or more copies of the target site sequence, less than 100% sequence identity to the target site sequence, or combinations thereof.

5. (canceled)

6. (canceled)

7. The method of claim 1, wherein the comparing step is performed without a reference sequence.

8. The method of claim 1, wherein the guide polynucleotide is designed for a target site sequence from a consensus sequence of a haplotype.

9. (canceled)

10. The method of claim 1, wherein the generating an intended gene edit at the target site in a nucleic acid using the designed guide polynucleotide in a Cas endonuclease complex.

11. (canceled)

12. (canceled)

13. (canceled)

14. The method of claim 13, the method further comprising: determining the presence or absence of the intended gene edit in the plant, mammal, virus, insect, fungus, or microorganism.

15. (canceled)

16. (canceled)

17. (canceled)

18. A method of creating a consensus sequence for a subject haplotype found in a population, the method comprising:
(a) sequencing a region of interest of two or more individuals of differing genotypes in a population to produce nucleotide sequence reads;
(b) aligning the nucleotide sequence reads to one or more subject sequences to identify nucleotide variations;
(c) using the nucleotide variations in the region of interest to define one or more haplotypes;
(d) assigning at least one individual from the population to the haplotypes in step (c);
(e) creating a profile for nucleotide variant frequencies for each common haplotype based on the nucleotide variations in the region of interest to generate common haplotype profiles;
(f) identifying whether there are breakpoints in the subject haplotype that correspond to the common haplotype profiles or combinations thereof;
(g) assigning those regions of the subject haplotype defined by the breakpoints to the corresponding two or more common haplotypes; and
(h) creating a consensus sequence for the haplotype assembled from the nucleotide sequence reads of the regions of the common haplotypes that the subject haplotype was assigned to from step (g).

19. The method of claim 18, wherein the subject haplotype is a rare haplotype.

20. (canceled)

21. (canceled)

22. (canceled)

23. The method of claim 18, wherein the subject haplotype sequence is matched to a profile comprising a consensus of sequence information from the common haplotype.

24. The method of claim 23, wherein the sequence information comprises the probability a nucleotide or amino acid is found at a certain position in the common haplotype sequence.

25. The method of claim 18, further comprising determining which common haplotype profiles fits the subject haplotype using a Viterbi algorithm adapted for comparing a single polynucleotide or amino acid sequence to a multiple alignment of a sequence family.

26. (canceled)

27. A method of characterizing two or more haplotypes found in a population, the method comprising:
(a) sequencing a defined region of interest in two or more individuals of differing genotypes in a population to produce nucleotide sequence reads;
(b) using nucleotide variations in the defined region to define two or more haplotypes;
(c) assembling the nucleotide sequence reads across the different genotypes into consensus sequences for the two or more haplotypes;
(d) comparing the haplotype consensus sequences to identify one or more additional nucleotide variations; and
(e) characterizing each haplotype based on the identified nucleotide variations in the region of interest.

28. The method of claim 27, further comprising:
(f) assigning at least one individual from the population to one or more haplotypes based on the nucleotide variations; and
(g) creating a haplotype consensus sequence assembled from the nucleotide sequence reads of the regions of the one or more individuals assigned in step (f).

29. The method of claim 18, wherein the certain nucleotide variation is a genetic marker, single nucleotide polymorphism (SNP), simple sequence repeat (SSR), microRNA, siRNA, quantitative trait loci (QTL), transgene, mRNA, or methylation pattern.

30. (canceled)

31. The method of claim 18, wherein the region of interest comprises a haplotype comprising genetically related and non-identical sequence.

32. (canceled)

33. The method of claim 18, wherein the individual comprises a homozygous genotype that differs from one or more subject sequences.

34. (canceled)

35. (canceled)

36. (canceled)

37. The method of claim 18, wherein the individuals comprise no more than a specified rate of missing sequence information.

38. The method of claim 37, wherein the specified rate of missing sequence information is 6% or less.

39. (canceled)

40. (canceled)

41. (canceled)

42. (canceled)

43. (canceled)

44. (canceled)


SEQUENCE LISTING (2 sequences)

SEQ ID NO: 1 - DNA, 3624 bp, Artificial Sequence
  consensus sequence
  misc_feature (96)..(118): n is a, c, g, or t
  misc_feature (124)..(124): n is a, c, g, or t
  misc_feature (128)..(128): n is a, c, g, or t
  misc_feature (135)..(135): n is a, c, g, or t
  misc_feature (149)..(149): n is a, c, g, or t
  misc_feature (153)..(153): n is a, c, g, or t
  misc_feature (166)..(166): n is a, c, g, or t
  misc_feature (206)..(229): n is a, c, g, or t
  misc_feature (236)..(251): n is a, c, g, or t
  misc_feature (261)..(262): n is a, c, g, or t
  misc_feature (264)..(265): n is a, c, g, or t
  misc_feature (267)..(270): n is a, c, g, or t
  misc_feature (278)..(278): n is a, c, g, or t
  misc_feature (287)..(287): n is a, c, g, or t
  misc_feature (291)..(291): n is a, c, g, or t
  misc_feature (299)..(299): n is a, c, g, or t
  misc_feature (366)..(366): n is a, c, g, or t
  misc_feature (389)..(389): n is a, c, g, or t
  misc_feature (402)..(402): n is a, c, g, or t
  misc_feature (438)..(438): n is a, c, g, or t
  misc_feature (457)..(457): n is a, c, g, or t
  misc_feature (474)..(474): n is a, c, g, or t
  misc_feature (503)..(503): n is a, c, g, or t
  misc_feature (506)..(507): n is a, c, g, or t
  misc_feature (509)..(510): n is a, c, g, or t
  misc_feature (512)..(512): n is a, c, g, or t
  misc_feature (518)..(518): n is a, c, g, or t
  misc_feature (520)..(520): n is a, c, g, or t
  misc_feature (593)..(593): n is a, c, g, or t
  misc_feature (595)..(595): n is a, c, g, or t
  misc_feature (597)..(598): n is a, c, g, or t
  misc_feature (602)..(603): n is a, c, g, or t
  misc_feature (605)..(605): n is a, c, g, or t
  misc_feature (609)..(609): n is a, c, g, or t
  misc_feature (624)..(624): n is a, c, g, or t
  misc_feature (628)..(643): n is a, c, g, or t
  misc_feature (647)..(647): n is a, c, g, or t
  misc_feature (663)..(664): n is a, c, g, or t
  misc_feature (697)..(713): n is a, c, g, or t
  misc_feature (778)..(778): n is a, c, g, or t
  misc_feature (790)..(790): n is a, c, g, or t
  misc_feature (799)..(799): n is a, c, g, or t
  misc_feature (831)..(831): n is a, c, g, or t
  misc_feature (873)..(894): n is a, c, g, or t
  misc_feature (920)..(935): n is a, c, g, or t
  misc_feature (1166)..(1197): n is a, c, g, or t
  misc_feature (1244)..(1245): n is a, c, g, or t
  misc_feature (1255)..(1256): n is a, c, g, or t
  misc_feature (1259)..(1259): n is a, c, g, or t
  misc_feature (1261)..(1261): n is a, c, g, or t
  misc_feature (1263)..(1263): n is a, c, g, or t
  misc_feature (1265)..(1268): n is a, c, g, or t
  misc_feature (1271)..(1281): n is a, c, g, or t
  misc_feature (1283)..(1284): n is a, c, g, or t
  misc_feature (1287)..(1289): n is a, c, g, or t
  misc_feature (1307)..(1307): n is a, c, g, or t
  misc_feature (1344)..(1344): n is a, c, g, or t
  misc_feature (1364)..(1364): n is a, c, g, or t
  misc_feature (1428)..(1445): n is a, c, g, or t
  misc_feature (1460)..(1460): n is a, c, g, or t
  misc_feature (1465)..(1465): n is a, c, g, or t
  misc_feature (1471)..(1471): n is a, c, g, or t
  misc_feature (1474)..(1474): n is a, c, g, or t
  misc_feature (1478)..(1478): n is a, c, g, or t
  misc_feature (1486)..(1486): n is a, c, g, or t
  misc_feature (1491)..(1491): n is a, c, g, or t
  misc_feature (1498)..(1498): n is a, c, g, or t
  misc_feature (1512)..(1512): n is a, c, g, or t
  misc_feature (1516)..(1516): n is a, c, g, or t
  misc_feature (1540)..(1540): n is a, c, g, or t
  misc_feature (1551)..(1551): n is a, c, g, or t
  misc_feature (1555)..(1555): n is a, c, g, or t
  misc_feature (1562)..(1562): n is a, c, g, or t
  misc_feature (1590)..(1590): n is a, c, g, or t
  misc_feature (1606)..(1606): n is a, c, g, or t
  misc_feature (1609)..(1626): n is a, c, g, or t
  misc_feature (1652)..(1653): n is a, c, g, or t
  misc_feature (1657)..(1657): n is a, c, g, or t
  misc_feature (1659)..(1659): n is a, c, g, or t
  misc_feature (1674)..(1674): n is a, c, g, or t
  misc_feature (1695)..(1695): n is a, c, g, or t
  misc_feature (1718)..(1718): n is a, c, g, or t
  misc_feature (1725)..(1727): n is a, c, g, or t
  misc_feature (1734)..(1734): n is a, c, g, or t
  misc_feature (1778)..(1778): n is a, c, g, or t
  misc_feature (1785)..(1786): n is a, c, g, or t
  misc_feature (1792)..(1792): n is a, c, g, or t
  misc_feature (1808)..(1808): n is a, c, g, or t
  misc_feature (1817)..(1818): n is a, c, g, or t
  misc_feature (1824)..(1824): n is a, c, g, or t
  misc_feature (1829)..(1829): n is a, c, g, or t
  misc_feature (1833)..(1833): n is a, c, g, or t
  misc_feature (1846)..(1861): n is a, c, g, or t
  misc_feature (1864)..(1864): n is a, c, g, or t
  misc_feature (1874)..(1874): n is a, c, g, or t
  misc_feature (1879)..(1879): n is a, c, g, or t
  misc_feature (1889)..(1889): n is a, c, g, or t
  misc_feature (1916)..(1916): n is a, c, g, or t
  misc_feature (1926)..(1926): n is a, c, g, or t
  misc_feature (1935)..(1935): n is a, c, g, or t
  misc_feature (1949)..(1949): n is a, c, g, or t
  misc_feature (1988)..(1988): n is a, c, g, or t
  misc_feature (1990)..(1990): n is a, c, g, or t
  misc_feature (1992)..(2000): n is a, c, g, or t
  misc_feature (2002)..(2002): n is a, c, g, or t
  misc_feature (2004)..(2004): n is a, c, g, or t
  misc_feature (2006)..(2006): n is a, c, g, or t
  misc_feature (2026)..(2026): n is a, c, g, or t
  misc_feature (2225)..(2241): n is a, c, g, or t
  misc_feature (2252)..(2268): n is a, c, g, or t
  misc_feature (2297)..(2297): n is a, c, g, or t
  misc_feature (2328)..(2328): n is a, c, g, or t
  misc_feature (2362)..(2362): n is a, c, g, or t
  misc_feature (2395)..(2396): n is a, c, g, or t
  misc_feature (2399)..(2513): n is a, c, g, or t
  misc_feature (2516)..(2708): n is a, c, g, or t
  misc_feature (2718)..(2720): n is a, c, g, or t
  misc_feature (2868)..(2868): n is a, c, g, or t
  misc_feature (2889)..(2916): n is a, c, g, or t
  misc_feature (2932)..(2932): n is a, c, g, or t
  misc_feature (2960)..(2960): n is a, c, g, or t
  misc_feature (2966)..(2966): n is a, c, g, or t
  misc_feature (2968)..(2994): n is a, c, g, or t
  misc_feature (3058)..(3058): n is a, c, g, or t
  misc_feature (3063)..(3064): n is a, c, g, or t
  misc_feature (3068)..(3068): n is a, c, g, or t
  misc_feature (3077)..(3080): n is a, c, g, or t
  misc_feature (3085)..(3085): n is a, c, g, or t
  misc_feature (3089)..(3089): n is a, c, g, or t
  misc_feature (3132)..(3132): n is a, c, g, or t
  misc_feature (3178)..(3199): n is a, c, g, or t
  misc_feature (3248)..(3257): n is a, c, g, or t
  misc_feature (3314)..(3329): n is a, c, g, or t
  misc_feature (3433)..(3434): n is a, c, g, or t
  misc_feature (3440)..(3441): n is a, c, g, or t
  misc_feature (3443)..(3443): n is a, c, g, or t
  misc_feature (3445)..(3445): n is a, c, g, or t
  misc_feature (3458)..(3458): n is a, c, g, or t
  misc_feature (3460)..(3460): n is a, c, g, or t
  misc_feature (3464)..(3464): n is a, c, g, or t
  misc_feature (3516)..(3531): n is a, c, g, or t
  misc_feature (3575)..(3575): n is a, c, g, or t
tagctacgtgcctgctcatgatcagaaccccagaccacgatctgcgtgctagcttcctcttgcactggcgatcccgtcgtgtcgtctctgcctctnnnnnnnnnnnnnnnnnnnnnnnacttgncacngcatgcnactccattgcgagngggnagaagaaaagggngagaagaccagagggaaaaacactacgcgcctatatatgnnnnnnnnnnnnnnnnnnnnnnnnagctagnnnnnnnnnnnnnnnnccgcagcttnnanncnnnnagcttaanaacattggntaantaataatnatcgtaacctcttgtacgtcccgactagctagtctaccaacccacccacgctgagctttcaatcgcncaaggagaaagaataatcgagangacggcacagganagctaaaacaaaagccttgtagttatggatgaagangaagatgatgataacacanaatatttaagtttggtntgtgtggctaagcagtggaaacacacacncanncnnangcatananagaaaaacaatgaaactttaaactagaacgacaagaagacgagagctaatattatggaagggtcttgatattncncnngaannangctncacgaactacacaanaaannnnnnnnnnnnnnnnatanttaaggttggcttttnnaaaagggcatgtgaaaaaaaaaggtagaacggnnnnnnnnnnnnnnnnnatcagatcgatgctctgcatatggagatcaggttaagacagcaattaatttgatgccgtcctatntatcggaaaacntgtcaaagngctgggagagacggtgtagtaggggggcatcnaaacattcacactaaaatggtgccatgtaggacactacttcnnnnnnnnnnnnnnnnnnnnnngagttgggagagttttttcggtacannnnnnnnnnnnnnnnctccactctaggcttcccacagtgggccagacaccttggcgctaggcttgacgatcctcttgggcctactgttgggcttgtgtcgctggtcacgcgggccttgtggcacacattgggatgactggcactctcttcctcgttgggcttgcggaaactgttggcgcaagcaaaaggctttgagacttcgcaggtagccgagtgttgcttgctggcatgtgtgatgtgattccnnnnnnnnnnnnnnnnnnnnnnnnnnnnnnnnacgggtgaccaatactaacatcgtattgtacctgctcgacaactatnngaagacattnnaantnanannnngannnnnnnnnnnanngannntactacatcggagttcanaaacaattgatgtatgctcctcggattgccacagtgngccgaatactttggcactangcttcacggtgctcctgggcctagcgttcagagtgagctctcgcttccaatgttgggcctgtgnnnnnnnnnnnnnnnnnntcagattggctaagnctatnttcggntgnttanctatctcngtatntatattnaaactccactctanaaantatagtataatatagtgatttgantgactatatgngtgnactgctngagacgacctaaccatgaggaaagaaanactttgaacatcaagnagnnnnnnnnnnnnnnnnnntcgatacgtaataacgtgtgtacgcnngtananaataaccaaaatatnttagaatgcatctagttaatnaaattaggttctttgagcctaancactgannntaagcantttgtttctagaccaaatttcatggtagttgggagcctacccanatttcannattaantgtgctattgaattgntgaaaatgnntgtgtntgtcntatncgacggataacgnnnnnnnnnnnnnnnntcnatgggcatgngcatngatatagatntgtacccactactagtatggtcgcagncggatattgnttgcaaccncagatatagtttcngggaaaaggattaggctcagctccatccctagaccccantngnnnnnnnnngngngngggggtctacccttcaaaangaaaaaaaactacacacagtgcatataagaagatgaatattccaaaattcagcagtcaagaagccctgataaactgtctggcatagctagtactttatacacttcaagaccaaaagaaatcactaagtacagattttagtgactcgtaagtacagatatcatcttacaaggcccagcccagcgacctattacacagccnnnnnnnnnnnnnnnnntcgggacacannnnnnnnnnnnnnnnngtgaagctctgctcgcagctgtccggctncttggacgttcgtgtggcagattcatctgtngtctcgtctcctgtgcttcctgggtagcttgtgnagtggagctgacatggtctgagcaggcttaaannttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgagcaggnnnaaaatttgctcgtagacgaggagtaccagcacagcacgttgcggatttctctgcctgtgaagtgcaacgtctaggattgtcacacgccttgtggtcgcgtcgcgtcgatgcggtggtgagcagagcagcaacagctgggcggcccaangttggcttccgtgtcttcgtnnnnnnnnnnnnnnnnnnnnnnnnnnnnagcagagagcggagancgagccgtgcacgggggaggtggtgtgnaagtgnannnnnnnnnnnnnnnnnnnnnnnnnnntgggcaacccaaaagtacccacgacaagcgaaggcgccaaagcgatccaagctccggaacgcancagcnnagcntcgcgtcgnnnnggagngcancagccacaagcagccgagaaccgaaccggtgggcgacgcgtcntgggacggacgcgggcgacgcttccaaacggggccacgtacgccgnnnnnnnnnnnnnnnnnnnnnnacgacaagccaaggcgaggcagcccccgatcgggaaagcgttttgggcnnnnnnnnnngcgtgcgggtcagtcgctggtgcgcagtgccggggggaacgggtatcgtggggggcnnnnnnnnnnnnnnnngtggcgagggccgagagcagcgcgcggccgggtcacgcaacgcgccccacgtactgccctccccctccgcgcgcgctagaaataccgaggcctggaccgggggnngccccnncncngtcacatccatcnancgancgatcgatcgccacagccaacaccacccgccgaggcgacgcgacagccgccnnnnnnnnnnnnnnnnctcactgccagccagtgaagggggagaagtgtactgctccgtcnaccagtgcgcgcaccgcccggcagggctgctcatctcgtcgacgaccag

SEQ ID NO: 2 - DNA, 3674 bp, Artificial Sequence
  waxy promoter consensus sequcens other strand
  misc_feature (96)..(118): n is a, c, g, or t
  misc_feature (124)..(124): n is a, c, g, or t
  misc_feature (128)..(128): n is a, c, g, or t
  misc_feature (135)..(135): n is a, c, g, or t
  misc_feature (149)..(149): n is a, c, g, or t
  misc_feature (153)..(153): n is a, c, g, or t
  misc_feature (166)..(166): n is a, c, g, or t
  misc_feature (206)..(229): n is a, c, g, or t
  misc_feature (236)..(251): n is a, c, g, or t
  misc_feature (261)..(262): n is a, c, g, or t
  misc_feature (264)..(265): n is a, c, g, or t
  misc_feature (267)..(270): n is a, c, g, or t
  misc_feature (278)..(278): n is a, c, g, or t
  misc_feature (287)..(287): n is a, c, g, or t
  misc_feature (291)..(291): n is a, c, g, or t
  misc_feature (299)..(299): n is a, c, g, or t
  misc_feature (366)..(366): n is a, c, g, or t
  misc_feature (389)..(389): n is a, c, g, or t
  misc_feature (402)..(402): n is a, c, g, or t
  misc_feature (438)..(438): n is a, c, g, or t
  misc_feature (457)..(457): n is a, c, g, or t
  misc_feature (474)..(474): n is a, c, g, or t
  misc_feature (503)..(503): n is a, c, g, or t
  misc_feature (506)..(507): n is a, c, g, or t
  misc_feature (509)..(510): n is a, c, g, or t
  misc_feature (512)..(512): n is a, c, g, or t
  misc_feature (518)..(518): n is a, c, g, or t
  misc_feature (520)..(520): n is a, c, g, or t
  misc_feature (593)..(593): n is a, c, g, or t
  misc_feature (595)..(595): n is a, c, g, or t
  misc_feature (597)..(598): n is a, c, g, or t
  misc_feature (602)..(603): n is a, c, g, or t
  misc_feature (605)..(605): n is a, c, g, or t
  misc_feature (609)..(609): n is a, c, g, or t
  misc_feature (624)..(624): n is a, c, g, or t
  misc_feature (628)..(643): n is a, c, g, or t
  misc_feature (647)..(647): n is a, c, g, or t
  misc_feature (663)..(664): n is a, c, g, or t
  misc_feature (697)..(713): n is a, c, g, or t
  misc_feature (751)..(763): n is a, c, g, or t
  misc_feature (828)..(828): n is a, c, g, or t
  misc_feature (840)..(840): n is a, c, g, or t
  misc_feature (849)..(849): n is a, c, g, or t
  misc_feature (881)..(881): n is a, c, g, or t
  misc_feature (923)..(944): n is a, c, g, or t
  misc_feature (970)..(985): n is a, c, g, or t
  misc_feature (1216)..(1247): n is a, c, g, or t
  misc_feature (1294)..(1295): n is a, c, g, or t
  misc_feature (1305)..(1306): n is a, c, g, or t
  misc_feature (1309)..(1309): n is a, c, g, or t
  misc_feature (1311)..(1311): n is a, c, g, or t
  misc_feature (1313)..(1313): n is a, c, g, or t
  misc_feature (1315)..(1318): n is a, c, g, or t
  misc_feature (1321)..(1331): n is a, c, g, or t
  misc_feature (1333)..(1334): n is a, c, g, or t
  misc_feature (1337)..(1339): n is a, c, g, or t
  misc_feature (1357)..(1357): n is a, c, g, or t
  misc_feature (1394)..(1394): n is a, c, g, or t
  misc_feature (1414)..(1414): n is a, c, g, or t
  misc_feature (1478)..(1495): n is a, c, g, or t
  misc_feature (1510)..(1510): n is a, c, g, or t
  misc_feature (1515)..(1515): n is a, c, g, or t
  misc_feature (1521)..(1521): n is a, c, g, or t
  misc_feature (1524)..(1524): n is a, c, g, or t
  misc_feature (1528)..(1528): n is a, c, g, or t
  misc_feature (1536)..(1536): n is a, c, g, or t
  misc_feature (1541)..(1541): n is a, c, g, or t
  misc_feature (1548)..(1548): n is a, c, g, or t
  misc_feature (1562)..(1562): n is a, c, g, or t
  misc_feature (1566)..(1566): n is a, c, g, or t
  misc_feature (1590)..(1590): n is a, c, g, or t
  misc_feature (1601)..(1601): n is a, c, g, or t
  misc_feature (1605)..(1605): n is a, c, g, or t
  misc_feature (1612)..(1612): n is a, c, g, or t
  misc_feature (1640)..(1640): n is a, c, g, or t
  misc_feature (1656)..(1656): n is a, c, g, or t
  misc_feature (1659)..(1676): n is a, c, g, or t
  misc_feature (1702)..(1703): n is a, c, g, or t
  misc_feature (1707)..(1707): n is a, c, g, or t
  misc_feature (1709)..(1709): n is a, c, g, or t
  misc_feature (1724)..(1724): n is a, c, g, or t
  misc_feature (1745)..(1745): n is a, c, g, or t
  misc_feature (1768)..(1768): n is a, c, g, or t
  misc_feature (1775)..(1777): n is a, c, g, or t
  misc_feature (1784)..(1784): n is a, c, g, or t
  misc_feature (1828)..(1828): n is a, c, g, or t
  misc_feature (1835)..(1836): n is a, c, g, or t
  misc_feature (1842)..(1842): n is a, c, g, or t
  misc_feature (1858)..(1858): n is a, c, g, or t
  misc_feature (1867)..(1868): n is a, c, g, or t
  misc_feature (1874)..(1874): n is a, c, g, or t
  misc_feature (1879)..(1879): n is a, c, g, or t
  misc_feature (1883)..(1883): n is a, c, g, or t
  misc_feature (1896)..(1911): n is a, c, g, or t
  misc_feature (1914)..(1914): n is a, c, g, or t
  misc_feature (1924)..(1924): n is a, c, g, or t
  misc_feature (1929)..(1929): n is a, c, g, or t
  misc_feature (1939)..(1939): n is a, c, g, or t
  misc_feature (1966)..(1966): n is a, c, g, or t
  misc_feature (1976)..(1976): n is a, c, g, or t
  misc_feature (1985)..(1985): n is a, c, g, or t
  misc_feature (1999)..(1999): n is a, c, g, or t
  misc_feature (2038)..(2038): n is a, c, g, or t
  misc_feature (2040)..(2040): n is a, c, g, or t
  misc_feature (2042)..(2050): n is a, c, g, or t
  misc_feature (2052)..(2052): n is a, c, g, or t
  misc_feature (2054)..(2054): n is a, c, g, or t
  misc_feature (2056)..(2056): n is a, c, g, or t
  misc_feature (2076)..(2076): n is a, c, g, or t
  misc_feature (2275)..(2291): n is a, c, g, or t
  misc_feature (2302)..(2318): n is a, c, g, or t
  misc_feature (2347)..(2347): n is a, c, g, or t
  misc_feature (2378)..(2378): n is a, c, g, or t
  misc_feature (2412)..(2412): n is a, c, g, or t
  misc_feature (2445)..(2446): n is a, c, g, or t
  misc_feature (2449)..(2563): n is a, c, g, or t
  misc_feature (2566)..(2758): n is a, c, g, or t
  misc_feature (2768)..(2770): n is a, c, g, or t
  misc_feature (2918)..(2918): n is a, c, g, or t
  misc_feature (2939)..(2966): n is a, c, g, or t
  misc_feature (2982)..(2982): n is a, c, g, or t
  misc_feature (3010)..(3010): n is a, c, g, or t
  misc_feature (3016)..(3016): n is a, c, g, or t
  misc_feature (3018)..(3044): n is a, c, g, or t
  misc_feature (3108)..(3108): n is a, c, g, or t
  misc_feature (3113)..(3114): n is a, c, g, or t
  misc_feature (3118)..(3118): n is a, c, g, or t
  misc_feature (3127)..(3130): n is a, c, g, or t
  misc_feature (3135)..(3135): n is a, c, g, or t
  misc_feature (3139)..(3139): n is a, c, g, or t
  misc_feature (3182)..(3182): n is a, c, g, or t
  misc_feature (3228)..(3249): n is a, c, g, or t
  misc_feature (3298)..(3307): n is a, c, g, or t
  misc_feature (3364)..(3379): n is a, c, g, or t
  misc_feature (3483)..(3484): n is a, c, g, or t
  misc_feature (3490)..(3491): n is a, c, g, or t
  misc_feature (3493)..(3493): n is a, c, g, or t
  misc_feature (3495)..(3495): n is a, c, g, or t
  misc_feature (3508)..(3508): n is a, c, g, or t
  misc_feature (3510)..(3510): n is a, c, g, or t
  misc_feature (3514)..(3514): n is a, c, g, or t
  misc_feature (3566)..(3581): n is a, c, g, or t
  misc_feature (3625)..(3625): n is a, c, g, or t
atcgatgcacggacgagtactagtcttggggtctggtgctagacgcacgatcgaaggagaacgtgaccgctagggcagcacagcagagacggagannnnnnnnnnnnnnnnnnnnnnntgaacngtgncgtacgntgaggtaacgctcncccntcttcttttcccnctcttctggtctccctttttgtgatgcgcggatatatacnnnnnnnnnnnnnnnnnnnnnnnntcgatcnnnnnnnnnnnnnnnnggcgtcgaanntnngnnnntcgaattnttgtaaccnattnattattantagcattggagaacatgcagggctgatcgatcagatggttgggtgggtgcgactcgaaagttagcgngttcctctttcttattagctctnctgccgtgtcctntcgattttgttttcggaacatcaatacctacttctncttctactactattgtgtnttataaattcaaaccanacacaccgattcgtcacctttgtgtgtgngtnngnntncgtatntntctttttgttactttgaaatttgatcttgctgttcttctgctctcgattataataccttcccagaactataangngnncttnntncgangtgcttgatgtgttntttnnnnnnnnnnnnnnnntatnaattccaaccgaaaannttttcccgtacactttttttttccatcttgccnnnnnnnnnnnnnnnnnatcagatcgatgctctgcatatggagatcaggttaagnnnnnnnnnnnnntagtctagctacgagacgtatacctctagtccaattctgtcgttaattaaactacggcaggatanatagccttttgnacagtttcncgaccctctctgccacatcatccccccgtagntttgtaagtgtgattttaccacggtacatcctgtgatgaagnnnnnnnnnnnnnnnnnnnnnnctcaaccctctcaaaaaagccatgtnnnnnnnnnnnnnnnngaggtgagatccgaagggtgtcacccggtctgtggaaccgcgatccgaactgctaggagaacccggatgacaacccgaacacagcgaccagtgcgcccggaacaccgtgtgtaaccctactgaccgtgagagaaggagcaacccgaacgcctttgacaaccgcgttcgttttccgaaactctgaagcgtccatcggctcacaacgaacgaccgtacacactacactaaggnnnnnnnnnnnnnnnnnnnnnnnnnnnnnnnntgcccactggttatgattgtagcataacatggacgagctgttgatanncttctgtaannttnantntnnnnctnnnnnnnnnnntnnctnnnatgatgtagcctcaagtntttgttaactacatacgaggagcctaacggtgtcacncggcttatgaaaccgtgatncgaagtgccacgaggacccggatcgcaagtctcactcgagagcgaaggttacaacccggacacnnnnnnnnnnnnnnnnnnagtctaaccgattcngatanaagccnacnaatngatagagncatanatataantttgaggtgagatntttnatatcatattatatcactaaactnactgatatacncacntgacganctctgctggattggtactcctttctttntgaaacttgtagttcntcnnnnnnnnnnnnnnnnnnagctatgcattattgcacacatgcgnncatntnttattggttttatanaatcttacgtagatcaattantttaatccaagaaactcggattngtgactnnnattcgtnaaacaaagatctggtttaaagtaccatcaaccctcggatgggtntaaagtnntaattnacacgataacttaacnacttttacnnacacanacagnatangctgcctattgcnnnnnnnnnnnnnnnnagntacccgtacncgtanctatatctanacatgggtgatgatcataccagcgtcngcctataacnaacgttggngtctatatcaaagncccttttcctaatccgagtcgaggtagggatctggggtnancnnnnnnnnncncncncccccagatgggaagttttncttttttttgatgtgtgtcacgtatattcttctacttataaggttttaagtcgtcagttcttcgggactatttgacagaccgtatcgatcatgaaatatgtgaagttctggttttctttagtgattcatgtctaaaatcactgagcattcatgtctatagtagaatgttccgggtcgggtcgctggataatgtgtcggnnnnnnnnnnnnnnnnnagccctgtgtnnnnnnnnnnnnnnnnncacttcgagacgagcgtcgacaggccgangaacctgcaagcacaccgtctaagtagacancagagcagaggacacgaaggacccatcgaacacntcacctcgactgtaccagactcgtccgaatttnnaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngactcgtccnnnttttaaacgagcatctgctcctcatggtcgtgtcgtgcaacgcctaaagagacggacacttcacgttgcagatcctaacagtgtgcggaacaccagcgcagcgcagctacgccaccactcgtctcgtcgttgtcgacccgccgggttncaaccgaaggcacagaagcannnnnnnnnnnnnnnnnnnnnnnnnnnntcgtctctcgcctctngctcggcacgtgccccctccaccacacnttcacntnnnnnnnnnnnnnnnnnnnnnnnnnnnacccgttgggttttcatgggtgctgttcgcttccgcggtttcgctaggttcgaggccttgcgtngtcgnntcgnagcgcagcnnnncctcncgtngtcggtgttcgtcggctcttggcttggccacccgctgcgcagnaccctgcctgcgcccgctgcgaaggtttgccccggtgcatgcggcnnnnnnnnnnnnnnnnnnnnnntgctgttcggttccgctccgtcgggggctagccctttcgcaaaacccgnnnnnnnnnncgcacgcccagtcagcgaccacgcgtcacggccccccttgcccatagcaccccccgnnnnnnnnnnnnnnnncaccgctcccggctctcgtcgcgcgccggcccagtgcgttgcgcggggtgcatgacgggagggggaggcgcgcgcgatctttatggctccggacctggcccccnncggggnngngncagtgtaggtagntngctngctagctagcggtgtcggttgtggtgggcggctccgctgcgctgtcggcggnnnnnnnnnnnnnnnngagtgacggtcggtcacttccccctcttcacatgacgaggcagntggtcacgcgcgtggcgggccgtcccgacgagtagagcagctgctggtc